(19) United States
(12) Patent Application Publication
(10) Pub. No.: US 2023/0416399 A1
(43) Pub. Date: Dec. 28, 2023

(54) METHODS FOR TREATING CANCER WITH ANTI-GD2/GD3 ANTIBODIES

(71) Applicant: Horacio Uri SARAGOVI, Montreal (CA)

(72) Inventor: Horacio Uri SARAGOVI, Montreal (CA)

(21) Appl. No.: 18/324,663

(22) Filed: May 26, 2023

Related U.S. Application Data
(60) Provisional application No. 63/346,465, filed on May 27, 2022.

(51) Int. Cl. C07K 16/30; A61K 39/395; A61K 45/06; A61P 35/00

(52) U.S. Cl. CPC C07K 16/3084; A61K 39/39558; A61K 39/3955; A61K 45/06; A61P 35/00

(57) ABSTRACT

The present disclosure provides methods for treatment and prevention of cancer comprising administration of certain monoclonal antibodies and antigen-binding fragments thereof that specifically bind to gangliosides (e.g., GD2 and/or GD3). There are also provided methods comprising administration of certain monoclonal antibodies and antigen-binding fragments thereof that specifically bind to gangliosides (e.g., GD2 and/or GD3) as an adjuvant therapy for cancer.

### CROSS-REFERENCE TO RELATED APPLICATIONS

This application claims the benefit of priority from U.S. provisional application No. 63/346,465 filed May 27, 2022, which is herein incorporated by reference in its entirety.

### REFERENCE TO AN ELECTRONIC SEQUENCE LISTING

This application contains a Sequence Listing which has been submitted in ASCII format via Patent Center and is hereby incorporated by reference in its entirety. The ASCII copy, created on Sep. 14, 2023, is named “40085-004 Sequence Listing Sep. 14 2023.xml” and is 167 KB.

### FIELD

The disclosure relates generally to the field of anti-cancer antibodies for targeting tumor cells. More specifically, the disclosure relates to methods of using antibodies specific for tumor marker gangliosides such as GD2 (anti-GD2 antibodies) or GD3 (anti-GD3 antibodies), and pharmaceutical compositions thereof, for the treatment and prevention of cancer.

### BACKGROUND

Cancer involves abnormal cell growth with the potential to invade or spread to other parts of the body. Despite decades of cancer research, cancer continues to cause a significant number of deaths (˜1,600 deaths per day in the U.S. in 2020). For example, ovarian cancer is the most lethal gynecologic cancer and the third cause of death among women.

Gangliosides are a family of >40 different sialic acid-containing glycosphingolipids, with an organization of two lipid tails embedded in the plasma membrane, a ceramide head, and an extracellular glycan tree. The glycan tree of each ganglioside is structurally unique, is conserved across all mammalian species, and defines each ganglioside. Gangliosides provide membrane integrity and modulate raft size, function and fluidity. Normal gangliosides such as GM1 are ubiquitous, but a subset of 20 are mainly expressed embryonically, are low/absent in normal cells, are and re-expressed at high levels in cancer.

Tumor ganglioside GD2 is expressed embryonically. After birth, its expression is low/absent (other than a low expression in ˜5% of sensory neurons), but GD2 is often re-expressed at very high levels in cancer. The tumor-associated gangliosides GD2 and GD3 and the normal gangljiside GM1 structures are shown in FIG. 1.

The GD2 and GD3 carbohydrate structure, and the GD2 re-expression patterns in cancer tissues and cell lines, are highly conserved from mice to humans. GD2 and GD3 in tumors provide significant functional advantages including pro-oncogenic, pro-angiogenic, and immune-evasion properties (Bartish, M. et al., Front Immunol 11, 564499, doi:10.3389/fimmu.2020.564499 (2020); Gagnon, M. & Saragovi, H. U., Expert Opinion in Therapeutic Patents 12, 1215-1223 (2002)). For example, GD2 expression in cells promotes ligand-independent activation of wild type receptor tyrosine kinases (e.g., EGF-R, TrkA, TrkB, PDGF-R, IGF1-R, MET), and wild type soluble kinases (e.g., Src, Lck), making expression of GD2 pro-oncogenic. GD2 and GD3 can interact with adhesion proteins, regulate membrane rafts, cell-cell and cell-matrix interactions, and enhance VEGF production and angiogenesis, providing the necessary blood supply that enables tumors to extravasate and metastasize. GD2 and GD3 can also suppress antigen presentation and inhibit T cell immunity locally and systemically, providing cancer with the ability to evade the immune system.

A common method of treating cancer involves using antibodies to attack tumor cells by specifically targeting tumor cell associated antigens. One specific example of this method involves using anti-GD2 antibodies targeted against GD2, which is highly expressed in certain tumor cells, such as glioblastoma, melanoma, Small-cell lung carcinoma, neuroblastoma, sarcoma, glioma, and ovarian cancer, and absent/very low in normal tissues. Indeed, GD2 was “ranked” by the NCI 12th in priority of all clinical cancer antigens (Cheever, M. A. et al., Clin Cancer Res 15, 5323-5337, doi:10.1158/1078-0432.CCR-09-0737 (2009)), and is a marker in ˜8% of all cancer related deaths in the U.S. (Regina Todeschini, A. & Hakomori, S. I., Biochimica et Biophysica Acta 1780, 421-433 (2008)). High expression of GD2 is associated with faster tumor progression, lower survival rates, and poor prognosis. When GD2 is expressed, its presence is uniform and homogeneous in all tumor nodules, and tumors do not lose GD2 after chemotherapy.

Although the etiological role of GD2 and GD3 in oncogenesis and immune suppression and its persistent and homogeneous expression in tumors make these glycolipids powerful therapeutic targets, exploiting these targets has been challenging. Despite more than 50 years of research and more than 35 years of clinical work, there are no truly effective clinical GD2-targeting or GD3-targeting agents. Development of anti-GD2 vaccines has been tried by many groups without success, due to low antibody titers and poor therapeutic efficacy. Very few selective anti-GD2 or anti-GD3 monoclonal antibodies (mAbs) are available. Glycolipids like GD2 are very poor immunogens, are not presented in the context of MHC/HLA, and possess heterogeneous lipid tails. Moreover, only subtle differences exist between normal GM1 and tumor GD2 (see FIG. 1), leading to concerns about tolerance or cross-reactive immunity.

The most immunogenic GD2 vaccine was tested clinically in advanced cutaneous melanoma (Daniotti, J. L., et al., Front Oncol 3, 306, doi:10.3389/fonc.2013.00306 (2013)), but the tumors progressed, and trials were discontinued. More recently, engineered chimeric antigen receptors (CAR) expressing an anti-GD2 mAb sequence in T or NK cells were used, alone and in combination with ICI blockade therapies, but showed no significant efficacy (Gargett, T. et al. Mol Ther 24, 1135-1149, doi:10.1038/mt.2016.63 (2016)). Other anti-GD2 CAR-T trials are ongoing, aiming to boost CAR-T efficacy, because commonly used anti-cancer drugs (including BRAF and MEK inhibitors for melanoma) arrest the proliferation of the anti-GD2 CAR-Ts. Two anti-GD2 monoclonal antibodies (mAbs) have achieved partial success clinically: anti-GD2 and a related anti-GD3 mAbs (Unituxin or Dinutuximab, 3F8 mAb and Ch14.18 mAb respectively), providing for clinical validation of the target. However, these mAbs have some efficacy only when used in combination with chemotherapy or linked to an effector agent, and received FDA-approval only for refractory Neuroblastoma because the disease has no other clinical options.

One serious problem of these therapies is a low Therapeutic Index which prevents their use in many indications. The therapies cause adverse events of high grade visceral pain, described as comparable to child-birth and not blocked by morphine, and optic nerve neuritis (Navid, F., et al., Curr Cancer Drug Targets 10, 200-209 (2010)). Because GD2 is expressed in a subset of nerve cells, pain and optic nerve neuritis are serious side effects of certain anti-GD2 antibody treatments (Kushner et al., J. Clin. Oncol., (2001; 19: 4189-94: Frost et al., Cancer (1997: 80: 317-33; Yu et al., J. Clin. Oncol., (1998; 16:2169-80).

Thus, although GD2 is a clinically validated anti-cancer target, anti-GD2 therapy has been extremely limited as it is difficult to raise selective immunity against glycolipids, and only a handful of anti-GD2 monoclonal antibodies (mAbs) exist. Few anti-GD2 mAbs have been FDA-approved, and only for very limited uses in cancer therapy due to poor efficacy and a low therapeutic index due to serious side effects (such as grade 4 pain, and optic nerve neuropathy in mice, rats, monkeys, and humans).

International Application No. PCT/US2021/05350 (WO/2022/076364) provides monoclonal antibodies and antigen-binding fragments thereof that specifically bind to gangliosides (e.g., GD2 and/or GD3), as well as compositions comprising same and methods of using same for diagnostic and prognostic purposes.

There is a need for antibodies specific for GD2 and/or GD3 that exhibit reduced side effects and/or a high therapeutic index while maintaining effectiveness in treating cancers that express the GD2 or GD3 glycolipid.

### SUMMARY

The present invention is based, at least in part, on the discovery that certain antibodies and antigen-binding fragments thereof that specifically bind to tumor gangliosides (e.g., GD2 and/or GD3) display reduced side effects, such as not causing pain, while maintaining efficacy for treating cancers. These antibodies therefore possess a high therapeutic index for cancer treatment and prevention. Accordingly, there are provided herein methods for treatment and prevention of cancer using the described antibodies and antigen-binding fragments thereof, as well as pharmaceutical compositions comprising same. These methods should allow the full exploitation of GD2 and GD3 as therapeutic anti-cancer targets.

The present invention is also based, at least in part, on the discovery that certain anti-GD2/GD3 antibodies and antigen-binding fragments thereof can potentiate the benefit of immune checkpoint inhibitor (ICI)-blockade therapy such as anti-PD-1. Accordingly, there are provided methods for using the described antibodies and antigen-binding fragments thereof, as well as pharmaceutical compositions comprising same, in combination with other anti-cancer therapies, such as ICI-blockade therapy.

In a first broad aspect, there are provided methods of treating or preventing cancer in a subject comprising administering antibodies, or antigen-binding fragments thereof, that are specific for a tumor ganglioside (e.g., GD2 and/or GD3) to the subject, such that the cancer is treated or prevented in the subject.

In a second broad aspect, there are provided methods of treating or preventing cancer in a subject comprising administering pharmaceutical compositions comprising antibodies, or antigen-binding fragments thereof, that are specific for a tumor ganglioside (e.g., GD2 and/or GD3) to the subject, such that the cancer is treated or prevented in the subject.

In a third broad aspect, there are provided methods of treating or preventing cancer in a subject comprising administering antibodies, or antigen-binding fragments thereof, that are specific for a tumor ganglioside (e.g., GD2 and/or GD3) to the subject in combination with one or more additional anti-cancer therapy, such that the cancer is treated or prevented in the subject.

In a fourth broad aspect, there are provided methods of treating or preventing cancer in a subject comprising administering pharmaceutical compositions comprising antibodies, or antigen-binding fragments thereof, that are specific for a tumor ganglioside (e.g., GD2 and/or GD3) to the subject in combination with one or more additional anti-cancer therapy, such that the cancer is treated or prevented in the subject.

In some embodiments of the third and fourth aspects, the additional anti-cancer therapy is an ICI-blockade therapy, such as, for example and without limitation, anti-PD1 or anti-PDL1. For example, administration of the anti-GD2/GD3 antibodies of the disclosure can act as an adjuvant therapy to render an ICI-resistant tumor responsive or sensitive to ICI. In other embodiments, the additional anti-cancer therapy is, for example and without limitation, chemotherapy, radiation therapy, hormone therapy, immunotherapy, targeted therapy, drug therapy, surgery or resection, or administration of another anti-cancer agent. The additional anti-cancer therapy may be administered before, after, or concomitantly with the anti-GD2/GD3 antibodies of the disclosure. The anti-GD2/GD3 antibodies may potentiate (e.g., increase, enhance) the additional anti-cancer therapy by, for example and without limitation, potentiating the therapeutic efficacy, stability, or penetration of the additional anti-cancer therapy in the subject.

In an aspect, there is provided a method of selecting and treating a subject suffering from an immune checkpoint inhibitor (ICI)-blockade therapy-resistant tumor or cancer, the method comprising the steps of: (a) selecting the subject as having an ICI-blockade therapy-resistant tumor or cancer based on the presence of GD2 and/or GD3 on the tumor or cancer cells; and (b) administering to the selected subject a therapeutically effective amount of an antibody specific for GD2 and/or GD3 or an antigen-binding fragment thereof. In some embodiments, the method further comprises administering to the selected subject an immune checkpoint inhibitor (ICI)-blockade therapy, such as for example and without limitation, an anti-PD1 or anti-PDL1 therapy, such as for example and without limitation, an antibody specific for PD1 or PDL1.

In an aspect, there is provided a method of selecting a subject suffering from an immune checkpoint inhibitor (ICI)-blockade therapy-resistant tumor or cancer, the method comprising the steps of: (a) obtaining a sample from the subject; (b) assaying the sample for binding to an antibody specific for GD2 and/or GD3, wherein the sample is respectively GD2+ and/or GD3+ if it binds the antibody; and (c) selecting the subject as suffering from an ICI-blockade therapy-resistant tumor or cancer if the sample is GD2+ and/or GD3+. In some embodiments, the method further comprises administering to the selected subject a therapeutically effective amount of an antibody specific for GD2 and/or GD3 or an antigen-binding fragment thereof. In some embodiments, the sample obtained from the subject is a tissue sample, e.g., comprising tumor or cancer cells, or a liquid tissue biopsy, e.g., blood or serum, and the like.

In some embodiments, there is provided a method of increasing the sensitivity of an ICI-resistant tumor to ICI-blockade therapy, the method comprising administration of the anti-GD2/GD3 antibodies of the disclosure, or a pharmaceutical composition thereof, to a subject before administration of the ICI-blockade therapy, or in combination with (co-administered with) the ICI-blockade therapy.

In some embodiments, there is provided a method of potentiating an anti-cancer therapy, the method comprising administration of the anti-GD2/GD3 antibodies of the disclosure, or a pharmaceutical composition thereof, to a subject before, after, or concomitantly with the administration of the anti-cancer therapy. The anti-cancer therapy may be, for example and without limitation, chemotherapy, radiation therapy, hormone therapy, immunotherapy, targeted therapy, drug therapy, surgery or resection, ICI-blockade therapy, or administration of another anti-cancer agent. The anti-GD2/GD3 antibody may potentiate the anti-cancer therapy by, for example and without limitation, potentiating the therapeutic efficacy, stability, or penetration of the additional anti-cancer therapy in the subject.

In some embodiments of methods of the present disclosure, antibodies or antigen-binding fragments specific for GD2 and/or GD3 are administered to the subject.

In some embodiments of methods of the present disclosure, antibodies or antigen-binding fragments specific for GD2 are administered to the subject.

In some embodiments of methods of the present disclosure, antibodies or antigen-binding fragments specific for GD3 are administered to the subject.

In some embodiments of methods of the present disclosure, antibodies or antigen-binding fragments specific for GD2 and GD3 are administered to the subject.

In some embodiments of methods of the present disclosure, the antibody or antigen-binding fragment thereof is a monoclonal antibody.

In some embodiments of methods of the present disclosure, antibodies or antigen-binding fragments specific for GD2 and/or GD3 are administered in the form of an antibody drug conjugate (ADC). For example, the antibody or antigen-binding fragment may be conjugated to an anti-cancer drug such as, for example and without limitation, a chemotherapeutic agent. An antibody or antigen-binding fragment may be conjugated to an anti-cancer drug directly or via a linker. In some embodiments, an antibody or antigen-binding fragment is conjugated to an anti-cancer drug via a cleavable linker.

In some embodiments, an antibody or antigen-binding fragment thereof for use in the methods of the present disclosure comprises one or more amino acid sequence listed in Table 1, or an amino acid sequence having at least about 75%, at least about 80%, at least about 85%, at least about 90%, at least about 95%, at least about 97%, at least about 98%, or at least about 99% identity thereto.

In some embodiments, an antibody or antigen-binding fragment thereof for use in the methods of the present disclosure comprises one or more amino acid sequence listed in Table 2, or an amino acid sequence having at least about 75%, at least about 80%, at least about 85%, at least about 90%, at least about 95%, at least about 97%, at least about 98%, or at least about 99% identity thereto.

In some embodiments, the antibody or antigen-binding fragment thereof for use in the methods of the present disclosure specifically binds to the carbohydrate portion of a ganglioside. In some embodiments, the ganglioside is (a) GD2, (b) GD3, or (c) GD2 and GD3.

In some embodiments, the antibody or antigen-binding fragment thereof for use in the methods of the present disclosure comprises: a) a combination of a heavy chain CDR1, CDR2, and CDR3 as set forth in Table 1, or a variant sequence thereof which differs by only one or two amino acids, or which has at least or about 85% sequence identity thereto; and/or b) a combination of a light chain CDR1, CDR2, and CDR3 as set forth in Table 1, or a variant sequence thereof which differs by only one or two amino acids, or which has at least or about 85% sequence identity thereto.

In some embodiments, the antibody or antigen-binding fragment thereof for use in the methods of the present disclosure comprises: a) a VH sequence as set forth in Table 2, or a variant sequence thereof which differs by only one or two amino acids, or which has at least or about 85% sequence identity thereto; and/or b) a VL sequence as set forth in Table 2, or a variant sequence thereof which differs by only one or two amino acids, or which has at least or about 85% sequence identity thereto.

In some embodiments, the antibody or antigen-binding fragment thereof for use in the methods of the present disclosure comprises: six CDR amino acid sequences selected from: a) SEQ ID NOs: 2, 4, 6, 8, 10, and 12 (clone 4), or a variant sequence thereof which differs by only one or two amino acids, or which has at least or about 85% sequence identity thereto; b) SEQ ID NOs: 14, 16, 18, 20, 22, and 24 (clone 6), or a variant sequence thereof which differs by only one or two amino acids, or which has at least or about 85% sequence identity thereto; c) SEQ ID NOs: 26, 28, 30, 32, 34, and 36 (clone 7), or a variant sequence thereof which differs by only one or two amino acids, or which has at least or about 85% sequence identity thereto; d) SEQ ID NOs: 38, 40, 42, 44, 46, and 48 (clone 8), or a variant sequence thereof which differs by only one or two amino acids, or which has at least or about 85% sequence identity thereto; e) SEQ ID NOs: 50, 52, 54, 56, 58, and 60 (clone 9), or a variant sequence thereof which differs by only one or two amino acids, or which has at least or about 85% sequence identity thereto; f) SEQ ID NOs: 62, 64, 66, 68, and 72 (clone 10), or a variant sequence thereof which differs by only one or two amino acids, or which has at least or about 85% sequence identity thereto; g) SEQ ID NOs: 74, 76, 78, 80, 82, and 84 (clone 13), or a variant sequence thereof which differs by only one or two amino acids, or which has at least or about 85% sequence identity thereto; h) SEQ ID NOs: 86, 88, 90, 92, 94, and 96 (clone 14), or a variant sequence thereof which differs by only one or two amino acids, or which has at least or about 85% sequence identity thereto; i) SEQ ID NOs: 98, 100, 102, 104, 106, and 108 (clone 15), or a variant sequence thereof which differs by only one or two amino acids, or which has at least or about 85% sequence identity thereto; j) SEQ ID NOs: 110, 112, 114, 116, 118, and 120 (clone 17), or a variant sequence thereof which differs by only one or two amino acids, or which has at least or about 85% sequence identity thereto; k) SEQ ID NOs: 122, 124, 126, 128, 130, and 132 (clone 18), or a variant sequence thereof which differs by only one or two amino acids, or which has at least or about 85% sequence identity thereto; and 1) SEQ ID NOs: 134, 136, 138, 140, 142, and 144 (clone 19), or a variant sequence thereof which differs by only one or two amino acids, or which has at least or about 85% sequence identity thereto.

In some embodiments, the antibody or antigen-binding fragment thereof for use in the methods of the present disclosure comprises: the VH and VL amino acid sequences selected from: a) SEQ ID NOs: 146 and 148, or a variant sequence thereof which differs by only one or two amino acids, or which has at least or about 85% sequence identity thereto; b) SEQ ID NOs: 150 and 152, or a variant sequence thereof which differs by only one or two amino acids, or which has at least or about 85% sequence identity thereto; c) SEQ ID NOs: 154 and 156, or a variant sequence thereof which differs by only one or two amino acids, or which has at least or about 85% sequence identity thereto; d) SEQ ID NOs: 158 and 160, or a variant sequence thereof which differs by only one or two amino acids, or which has at least or about 85% sequence identity thereto; e) SEQ ID NOs: 162 and 164, or a variant sequence thereof which differs by only one or two amino acids, or which has at least or about 85% sequence identity thereto; f) SEQ ID NOs: 166 and 168, or a variant sequence thereof which differs by only one or two amino acids, or which has at least or about 85% sequence identity thereto; g) SEQ ID NOs: 170 and 172, or a variant sequence thereof which differs by only one or two amino acids, or which has at least or about 85% sequence identity thereto; h) SEQ ID NOs: 174 and 176, or a variant sequence thereof which differs by only one or two amino acids, or which has at least or about 85% sequence identity thereto; i) SEQ ID NOs: 178 and 180, or a variant sequence thereof which differs by only one or two amino acids, or which has at least or about 85% sequence identity thereto; j) SEQ ID NOs: 182 and 184, or a variant sequence thereof which differs by only one or two amino acids, or which has at least or about 85% sequence identity thereto; k) SEQ ID NOs: 186 and 188, or a variant sequence thereof which differs by only one or two amino acids, or which has at least or about 85% sequence identity thereto; and 1) SEQ ID NOs: 190 and 192, or a variant sequence thereof which differs by only one or two amino acids, or which has at least or about 85% sequence identity thereto.

In some embodiments, the antibody or antigen-binding fragment thereof for use in the methods of the present disclosure comprises an antibody or antigen-binding fragment thereof which is chimeric, humanized, camelid, llama, composite, murine, or human.

In some embodiments, the antibody or antigen-binding fragment thereof for use in the methods of the present disclosure comprises an immunoglobulin heavy chain constant domain selected from the group consisting of IgG, IgG1, IgG2, IgG2A, IgG2B, IgG3, IgG4, IgA, IgM, IgD, and IgE constant domains.

In some embodiments, the antibody or antigen-binding fragment thereof for use in the methods of the present disclosure comprises comprises an effector domain, comprises an Fc domain, and/or is selected from the group consisting of Fv, F(ab′)2, Fab′, dsFv, scFv, sc(Fv)2, and diabody fragments.

In some embodiments, the antibody or antigen-binding fragment thereof for use in the methods of the present disclosure comprises an immunoglobulin heavy and/or light chain selected from the immunoglobulin heavy and light chain sequences listed in Table 2.

In some embodiments, the antibody or antigen-binding fragment thereof for use in the methods of the present disclosure comprises clone 4, clone 6, clone 17, or clone 19.

In one embodiment, the antibody or antigen-binding fragment thereof for use in the methods of the present disclosure comprises or consists of clone 4. In an embodiment, the antibody or antigen-binding fragment thereof for use in the methods of the present disclosure comprises or consists of the amino acid sequence set forth in SEQ ID NO: 146 (VH) and/or SEQ ID NO: 148 (VL), or a variant sequence thereof which differs by only one or two amino acids, or which has at least or about 85% sequence identity thereto.

In one embodiment, the antibody or antigen-binding fragment thereof for use in the methods of the present disclosure comprises or consists of clone 6. In an embodiment, the antibody or antigen-binding fragment thereof for use in the methods of the present disclosure comprises or consists of the amino acid sequence set forth in SEQ ID NO: 150 (VH) and/or SEQ ID NO: 152 (VL), or a variant sequence thereof which differs by only one or two amino acids, or which has at least or about 85% sequence identity thereto.

In one embodiment, the antibody or antigen-binding fragment thereof for use in the methods of the present disclosure comprises or consists of clone 17. In an embodiment, the antibody or antigen-binding fragment thereof for use in the methods of the present disclosure comprises or consists of the amino acid sequence set forth in SEQ ID NO: 182 (VH) and/or SEQ ID NO: 184 (VL), or a variant sequence thereof which differs by only one or two amino acids, or which has at least or about 85% sequence identity thereto.

In one embodiment, the antibody or antigen-binding fragment thereof for use in the methods of the present disclosure comprises or consists of clone 19. In an embodiment, the antibody or antigen-binding fragment thereof for use in the methods of the present disclosure comprises or consists of the amino acid sequence set forth in SEQ ID NO: 190 (VH) and/or SEQ ID NO: 192 (VL), or a variant sequence thereof which differs by only one or two amino acids, or which has at least or about 85% sequence identity thereto.

In one embodiment, the antibody or antigen-binding fragment thereof for use in the methods of the present disclosure comprises one or more CDR amino acid sequences selected from SEQ ID NOs: 2, 4, 6, 8, 10, and 12, or a variant sequence thereof which differs by only one or two amino acids, or which has at least or about 85% sequence identity thereto.

In one embodiment, the antibody or antigen-binding fragment thereof for use in the methods of the present disclosure comprises one or more CDR amino acid sequences selected from SEQ ID NOs: 14, 16, 18, 20, 22, and 24, or a variant sequence thereof which differs by only one or two amino acids, or which has at least or about 85% sequence identity thereto.

In one embodiment, the antibody or antigen-binding fragment thereof for use in the methods of the present disclosure comprises one or more CDR amino acid sequences selected from SEQ ID NOs: 110, 112, 114, 116, 118, and 120, or a variant sequence thereof which differs by only one or two amino acids, or which has at least or about 85% sequence identity thereto.

In one embodiment, the antibody or antigen-binding fragment thereof for use in the methods of the present disclosure comprises one or more CDR amino acid sequences selected from SEQ ID NOs: 134, 136, 138, 140, 142, and 144, or a variant sequence thereof which differs by only one or two amino acids, or which has at least or about 85% sequence identity thereto.

In some embodiments, the composition for use in the methods of the present disclosure comprises a nucleic acid molecule that encodes: a) a polypeptide comprising an amino acid sequence listed in Table 1 and/or Table 2; b) a polypeptide comprising an amino acid sequence with at least or about 85% identity to an amino acid sequence listed in Table 1 and/or Table 2; and/or c) a monoclonal antibody or antigen-binding fragment thereof for use in the methods of the present disclosure, as described herein. In some embodiments, the composition comprises a vector comprising the isolated nucleic acid as described herein.

In some embodiments of methods of the present disclosure, the cancer is selected from the group consisting of neuroblastoma, lymphoma, leukemia, melanoma, glioma, small cell lung cancer, breast carcinoma, ovarian cancer, soft tissue sarcomas, osteosarcoma, Ewing's sarcoma, desmoplastic round cell tumor, rhabdomyosarcoma, retinoblastoma, non-small cell lung cancer, renal cell cancer, Wilms tumor, prostate cancer, gastric cancer, endometrial cancer, pancreatic cancer, and colon cancer. In some embodiments, the cancer is selected from the group consisting of neuroblastoma, lymphoma, leukemia, melanoma, glioma, small cell lung cancer, breast carcinoma, ovarian cancer, soft tissue sarcomas, osteosarcoma, Ewing's sarcoma, desmoplastic round cell tumor, rhabdomyosarcoma, and retinoblastoma.

In some embodiments, the cancer is glioblastoma, melanoma, Small-cell lung carcinoma, neuroblastoma, sarcoma, glioma, or ovarian cancer.

Since the carbohydrate moiety of a ganglioside (e.g., GD2) is highly conserved in all mammals, it is expected that the anti-human GD2/GD3 antibodies described herein can be used to treat cancer in all mammals. In some embodiments of methods of the present disclosure therfore, the subject is a mammal. In an embodiment, the subject is a human. In other embodiments, the subject is an animal, e.g., a pet (dog, cat, mouse, rat, etc.), livestock (cow, sheep, goat, horse, etc.), an animal model of cancer, etc.

In methods of the present disclosure, the antibodies or the pharmaceutical compositions thereof may be administered using any suitable route of administration. Non-limiting routes of administration include parenteral, e.g., intravenous, intradermal, subcutaneous, intraperitoneal; oral, e.g., inhalation, swallowing; transdermal or topical; transmucosal; and rectal administration.

In certain aspects, there are provided therapeutic and prophylactic methods as described in detail herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the invention and to show more clearly how it may be carried into effect, reference will now be made by way of example to the accompanying drawings, which illustrate aspects and features according to embodiments of the present invention, and in which:

FIG. 1 shows a schematic diagram of ganglioside structures, including GM1, GD2 and GD3 gangliosides. Each geometric shape represents a type of sugar. The purple rhomboid shape represents sialic acid(s) that define the di-sialo-ganglioside GD2 or GD3. The glycan tree is linked to a ceramide and two lipid tails embedded in the outer leaflet of the membrane. The surface glycan structures define each ganglioside and are conserved from mice to humans. Normal GM1 and tumor GD2 differ by just 2 sugars, and GD2 and GD3 differ from each other by 1 sugar.

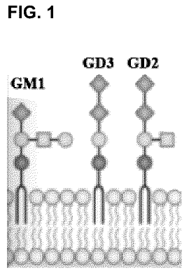

FIG. 2 shows characterization of monoclonal antibodies (mAbs) using flow cytometry. Binding assays were done by quantitative flow cytometry using EL4-GD2⁺ (expressing GD2 but not expressing GD3) or EL4-GD3⁺ cells (expressing GD3 but not expressing GD2) or Jurkat cells (expressing GM1 but neither GD2 nor GD3). Histograms show the mean channel fluorescence. (A) shows mAb clone 15 and clone 7 (blue) bind specifically to cells expressing GD2 and not to negative control (mouse IgG)(pink). (B) shows mAb clone 15 and clone 7 do not bind to cells lacking GD2. (C) shows mAb clone 19 (blue) binds to cells expressing GD2. Negative controls are mouse IgG (dashes) and mAb clone 6 (gray). (D) shows mAb clone 6 (blue) binds specifically to cells expressing GD3 (but not expressing GD2). Positive control is anti-GD3 (red, commercial source). Negative controls are mouse IgG (dashes) and anti-GD2 mAb clone 19 (gray).

FIG. 3 shows characterization of mAbs by ELISA. ELISA binding studies were done using biotinylated mAb clone 19 followed by streptavidin conjugated to HRP. The graph shows the raw Optical Density (O.D.₄₅₀) values for a GD2 standard curve (r² 0.9948).

FIG. 4 shows mAbs reduced primary tumors and extended life-span, and synergized with PD1-targeted therapies, in a mouse model. (A) shows anti-GD2 mAbs exhibited high efficacy against EL4 primary tumors in vivo, in a therapeutic paradigm with pre-existing tumor subcutaneously; n=6/group. (B) shows % Survival at indicated days post-implantation of EL4 tumors, n=5/group. The mAb clone 4 was tested in combination with PD1, and showed potentiation of therapy, with 60% long-term survivors in this very aggressive syngeneic tumor model.

FIG. 5 shows mAb therapy in a melanoma model. (A) shows the experimental paradigm and timeline. (B) shows primary tumor volume at days 27, 30 and 32, mAb clone 4 reduced primary tumor growth. (C) shows the weight of the lymph nodes, indicating that mAb clone 4 reduced metastasis to the lymph nodes; the horizontal line shows the mass/size of a normal naïve lymph node.

### DETAILED DESCRIPTION

The present disclosure provides methods of using certain monoclonal antibodies, and antigen-binding fragments thereof, that specifically bind to a tumor ganglioside (e.g., GD2 and/or GD3), as well as immunoglobulins, polypeptides, and nucleic acids thereof, for the treatment and prevention of cancer. There are also provided methods of using pharmaceutical compositions comprising the monoclonal antibodies, and antigen-binding fragments thereof, for the treatment and prevention of cancer. Further provided herein are methods of using the monoclonal antibodies, and antigen-binding fragments thereof, that specifically bind to a ganglioside (e.g., GD2 or GD3), as well as immunoglobulins, polypeptides, and nucleic acids thereof, as an adjuvant therapy for cancer, e.g., in combination with one or more additional anti-cancer therapy such as immune checkpoint inhibitor (ICI) therapy.

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The articles “a” and “an” are used herein to refer to one or to more than one (i.e. to at least one) of the grammatical object of the article. By way of example, “an element” means one element or more than one element.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The present description refers to a number of chemical terms and abbreviations used by those skilled in the art. Nevertheless, definitions of selected terms are provided for clarity and consistency.

As used herein, the term “composite antibody” refers to an antibody which has variable regions comprising germline or non-germline immunoglobulin sequences from two or more unrelated variable regions. Additionally, the term “composite, human antibody” refers to an antibody which has constant regions derived from human germline or non-germline immunoglobulin sequences and variable regions comprising human germline or non-germline sequences from two or more unrelated human variable regions.

The terms “CDR”, and its plural “CDRs,” refer to a complementarity determining region (CDR) of which three make up the binding character of a light chain variable region (CDR-L1, CDR-L2 and CDR-L3) and three make up the binding character of a heavy chain variable region (CDR-H1, CDR-H2 and CDR-H3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat, Chothia, and/or MacCallum et al., (Kabat et al., in “Sequences of Proteins of Immunological Interest,” 5^{th} Edition, U.S. Department of Health and Human Services, 1992; Chothia et al. (1987) J. Mol. Biol. 196, 901; and MacCallum et al., J. Mol. Biol. (1996) 262, 732, each of which is incorporated by reference in its entirety).

As used herein, the term “Fc region” is used to describe a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the present disclosure include human IgG1, IgG2 (IgG2A, IgG2B), IgG3 and IgG4.

As used herein, the term “anti-GD2/GD3 antibody” is used to encompass antibodies that are specific for GD2 and/or GD3, i.e., the term includes antibodies specific for GD2 (anti-GD2 antibodies), antibodies specific for GD3 (anti-GD3 antibodies), and antibodies specific for GD2 and GD3 (anti-GD2 and GD3 antibodies).

As used herein, the term “specific for” means that a polypeptide (such as an antibody) specifically interacts with a given target(s). Specific binding is believed to be effected by specific motifs in the amino acid sequence of a polypeptide. Thus, binding is achieved as a result of their primary, secondary and/or tertiary structure as well as the result of secondary modifications of said structures. The specific interaction of the target-interaction-site with its specific target may result in a simple binding of said site to the target, or may alternatively or additionally result in the initiation of a signal, e.g. due to the induction of a change of the conformation of the target, an oligomerization of the target, or may block the target from performing another activity, such as binding to an endogenous molecule. Generally, binding is considered specific when the binding affinity is about 10⁻¹² to 10⁻⁹ M, 10⁻¹² to 10⁻¹⁹ M, 10⁻¹¹ to 10⁻⁹ M, or of about 10⁻¹¹ to 10⁻⁹ M. The terms “(specifically) binds to”, “(specifically) recognizes”, “specific for”, “is (specifically) directed to”, and “(specifically) reacts with” are used interchangeably herein.

As used herein, the term “K_{D}” is intended to refer to the dissociation equilibrium constant of a particular antibody-antigen interaction. The binding affinity of antibodies of the disclosure may be measured or determined by standard antibody-antigen assays, for example, competitive assays, saturation assays, or standard immunoassays such as ELISA or RIA.

The term “minimal residual disease” is art recognized, and is used to describe a small number of cancer cells in the body during or after cancer treatment, when the patient is in remission. The number of remaining cells may be so small that they do not cause any physical signs or symptoms and often cannot even be detected through traditional methods. It is a major cause of relapse of cancer.

The term “adjuvant therapy” is used herein to refer to a treatment given before, after, or at the same time as, the primary anti-cancer treatment. Examples of adjuvant therapy can include, without limitation, chemotherapy, radiation therapy, and hormone therapy. Adjuvant therapies are often given to help reduce the size of a tumor, kill cancer cells that have spread, destroy remaining cancer cells, or potentiate the primary anti-cancer treatment.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The term “preventing” is art-recognized, and when used in relation to a condition, such as a viral/bacterial infection or a disease such as cancer is well understood in the art, and includes administration of a treatment, e.g., a composition which reduces the frequency of, or delays the onset of, symptoms of a medical condition in a subject relative to a subject which does not receive the treatment. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The term “remission” is art recognized, and refers to a condition in which the signs and symptoms of the cancer are reduced.

As used herein, “subject” refers to any healthy animal, mammal or human, or any animal, mammal or human afflicted with a cancer. The term “subject” is interchangeable with “patient”. The term “non-human animal” includes all vertebrates, e.g., mammals and non-mammals, such as non-human primates, sheep, dog, cow, chickens, amphibians, reptiles, etc.

### Antigen-Binding Proteins

Antigen-binding proteins for use in the methods of the disclosure are proteins that bind to a ganglioside (e.g., GD2 or GD3). In various embodiments, the antigen binding proteins bind to the carbohydrate portion of the ganglioside (e.g., GD2 or GD3). The antigen-binding proteins for use in the methods of the present disclosure can take any one of many forms of antigen-binding proteins known in the art. In various embodiments, the antigen-binding proteins of the present disclosure take the form of an antibody, or antigen-binding antibody fragment, or an antibody protein product.

In various embodiments of the present disclosure, the antigen-binding protein comprises, consists essentially of, or consists of an antibody. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4th ed. Elsevier Science Ltd./Garland Publishing, (1999)).

Unless otherwise specified here within, antibody or antibodies broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. Framework or FR residues are those variable-domain residues other than the hypervariable residues as herein indicated. The variable regions of the heavy and light chains contain a binding domain that interacts with an antigen.

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N. I. H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra). In a related embodiment, the residues of the framework are altered. The heavy chain framework regions which can be altered lie within regions designated H-FR1, H-FR2, H-FR3 and H-FR4, which surround the heavy chain CDR residues, and the residues of the light chain framework regions which can be altered lie within the regions designated L-FR1, L-FR2, L-FR3 and L-FR4, which surround the light chain CDR residues. An amino acid within the framework region may be replaced, for example, with any suitable amino acid identified in a human framework or human consensus framework.

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in various embodiments, the antibody is an antibody of isotype IgA, IgD, IgE, IgG, or IgM, including any one of IgG1, IgG2, IgG3 or IgG4. In various aspects, the antibody comprises a constant region comprising one or more amino acid modifications, relative to the naturally-occurring counterpart, in order to improve half-life/stability or to render the antibody more suitable for expression/manufacturability. In various instances, the antibody comprises a constant region wherein the C-terminal Lys residue that is present in the naturally-occurring counterpart is removed or clipped.

The antibody can be a monoclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antigen-binding protein is an antibody, such as a human antibody. In certain aspects, the antigen-binding protein is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence. Information, including sequence information for human antibody heavy and light chain constant regions is publicly available through the Uniprot database as well as other databases well-known to those in the field of antibody engineering and production. For example, the IgG2 constant region is available from the Uniprot database as Uniprot number P01859, incorporated herein by reference.

Antibody as used herein also includes antigen-binding portion of an antibody. The antigen-binding portion refers to one or more fragments of an antibody that retain the ability to specifically bind to an antigen (e.g., a ganglioside). It has been shown that the antigen-binding function of an antibody can be performed by fragments of a full-length antibody. Examples of binding fragments encompassed within the antigen-binding portion of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the antigen-binding portion of an antibody.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)2 fragment and a pFc′ fragment. In various aspects of the present disclosure, the antigen-binding protein of the present disclosure is an antigen-binding fragment of an antibody (a.k.a., antigen-binding antibody fragment, antigen-binding fragment, antigen-binding portion). In various instances, the antigen-binding antibody fragment is a Fab fragment or a F(ab′)2 fragment.

The architecture of antibodies has been exploited to create a growing range of alternative antibody formats that spans a molecular-weight range of at least or about 12-150 kDa and has a valency (n) range from monomeric (n=1), to dimeric (n=2), to trimeric (n=3), to tetrameric (n=4), and potentially higher; such alternative antibody formats are referred to herein as “antibody protein products”. Antibody protein products include those based on the full antibody structure and those that mimic antibody fragments which retain full antigen-binding capacity, e.g., scFvs, Fabs and VHH/VH (discussed below). The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, bispecific antibody (BsAb) fragments, bispecific fusion proteins, and BsAb conjugates. See, e.g., Spiess et al., Molecular Immunology 67(2) Part A: 97-106 (2015).

In various aspects, the antigen-binding protein for use in the methods of the present disclosure comprises, consists essentially of, or consists of any one of these antibody protein products. In various aspects, the antigen-binding protein of the present disclosure comprises, consists essentially of, or consists of any one of a Fab VHH/VH, Fv fragment, ds-scFv, scFab, Fv, F(ab′)2, Fab′, dsFv, scFv, sc(Fv)2, dimeric antibody, multimeric antibody (e.g., a diabody, triabody, tetrabody), miniAb, peptibody VHH/VH of camelid heavy chain antibody, sdAb, diabody; a triabody; and a tetrabody.

In various instances, the antigen-binding protein for use in the methods of the present disclosure is an antibody protein product in monomeric form, or polymeric, oligomeric, or multimeric form.

In certain aspects, provided herein is a monoclonal antibody, or antigen-binding fragment thereof, wherein the monoclonal antibody specifically binds to the carbohydrate portion of a ganglioside. In some embodiments, the ganglioside is (a) GD2, (b) GD3, or (c) GD2 and GD3.

In various embodiments, an anti-ganglioside antibody (against GD2 or GD3) or antibody variant thereof is selected from the group consisting of a human antibody, a humanized antibody, a chimeric antibody, a monoclonal antibody, a recombinant antibody, an antigen-binding antibody fragment, a single chain antibody, a camelid antibody, a llama antibody, a monomeric antibody, a diabody, a triabody, a tetrabody, Fv, F(ab′)2, Fab′, dsFv, scFv, sc(Fv)2, an IgG1 antibody, an IgG2 antibody, an IgG3 antibody, and an IgG4 antibody.

In certain aspects, an antibody for use in the methods of the present disclosure is a monoclonal antibody, or antigen-binding fragment thereof, wherein the monoclonal antibody comprises a) a heavy chain complementarity determining region (CDR) sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a heavy chain CDR sequence selected from the group consisting of the sequences listed in Table 1; and/or b) a light chain CDR sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a light chain CDR sequence selected from the group consisting of the sequences listed in Table 1.

In certain aspects, an antibody for use in the methods of the present disclosure is a monoclonal antibody, or antigen-binding fragment thereof, wherein the monoclonal antibody comprises a) a heavy chain variable domain (VH) with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a VH sequence selected from the group consisting of the VH sequences listed in Table 2; and/or b) a light chain variable domain (VL) sequence with at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identity to a VL sequence selected from the group consisting of the VL sequences listed in Table 2.

In certain aspects, an antibody for use in the methods of the present disclosure is a monoclonal antibody, or antigen-binding fragment thereof, wherein the monoclonal antibody, or antigen-binding fragment thereof, comprises a) a combination of a heavy chain CDR1, CDR2, and CDR3 as set forth in Table 1, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or b) a combination of a light chain CDR1, CDR2, and CDR3 as set forth in Table 1, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In certain aspects, an antibody for use in the methods of the present disclosure is a monoclonal antibody, or antigen-binding fragment thereof, wherein the monoclonal antibody, or antigen-binding fragment thereof, comprises: a) a VH sequence as set forth in Table 2, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and/or b) a VL sequence as set forth in Table 2, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

In some embodiments, an antibody for use in the methods of the present disclosure is a monoclonal antibody, or antigen-binding fragment thereof, wherein the monoclonal antibody comprises a) a VH sequence selected from the group consisting of the VH sequences listed in Table 2; and/or b) a VL sequence selected from the group consisting of the VL sequences listed in Table 2.

In some embodiments, an antibody for use in the methods of the present disclosure is the monoclonal antibody or antigen-binding fragment thereof comprising six CDR amino acid sequences selected from: a) SEQ ID NOs: 2, 4, 6, 8, 10, and 12 (clone 4), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; b) SEQ ID NOs: 14, 16, 18, 20, 22, and 24 (clone 6), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; c) SEQ ID NOs: 26, 28, 30, 32, 34, and 36 (clone 7), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; d) SEQ ID NOs: 38, 40, 42, 44, 46, and 48 (clone 8), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; e) SEQ ID NOs: 50, 52, 54, 56, 58, and 60 (clone 9), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; f) SEQ ID NOs: 62, 64, 66, 68, 70, and 72 (clone 10), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; g) SEQ ID NOs: 74, 76, 78, 80, 82, and 84 (clone 13), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; h) SEQ ID NOs: 86, 88, 92, 94, and 96 (clone 14), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; i) SEQ ID NOs: 98, 100, 102, 104, 106, and 108 (clone 15), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; j) SEQ ID NOs: 110, 112, 114, 116, 118, and 120 (clone 17), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; k) SEQ ID NOs: 122, 124, 126, 128, 130, and 132 (clone 18), or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and 1) SEQ ID NOs: 134, 136, 138, 140, 142, and 144 (clone 19), or a variant sequence thereof which differs by only one or two amino acids, or which has at least or about 85% sequence identity.

In some embodiments, an antibody for use in the methods of the present disclosure is the monoclonal antibody or antigen-binding fragment thereof comprising the VH and VL amino acid sequences selected from: a) SEQ ID NOs: 146 and 148, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; b) SEQ ID NOs: 150 and 152, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; c) SEQ ID NOs: 154 and 156, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; d) SEQ ID NOs: 158 and 160, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; e) SEQ ID NOs: 162 and 164, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; f) SEQ ID NOs: 166 and 168, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; g) SEQ ID NOs: 170 and 172, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; h) SEQ ID NOs: 174 and 176, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; i) SEQ ID NOs: 178 and 180, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; j) SEQ ID NOs: 182 and 184, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; k) SEQ ID NOs: 186 and 188, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity; and 1) SEQ ID NOs: 190 and 192, or a variant sequence thereof which differs by only one or two amino acids, or which has at least or about 30%, 35%, 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% sequence identity.

Numerous embodiments are further provided that can be applied to any aspect of the present technology described herein. For example, in some embodiments, an antibody for use in the methods of the present disclosure is a monoclonal antibody, or antigen-binding fragment thereof, which is chimeric, humanized, composite, murine, or human. In some embodiments, a monoclonal antibody, or antigen-binding fragment thereof, comprises an immunoglobulin heavy chain constant domain selected from the group consisting of IgG, IgG1, IgG2, IgG2A, IgG2B, IgG3, IgG4, IgA, IgM, IgD, and IgE constant domains. In some embodiments, a monoclonal antibody, or antigen-binding fragment thereof, is detectably labeled, comprises an effector domain, comprises an Fc domain, and/or is selected from the group consisting of Fv, F(ab′)2, Fab′, dsFv, scFv, sc(Fv)2 fragments, diabodies, bivalent, multivalent, and bifunctional engineered constructs. In some embodiments, a monoclonal antibody, or antigen-binding fragment thereof, is obtainable from hybridoma. In some embodiments, a monoclonal antibody, or antigen-binding fragment thereof, specifically binds a ganglioside (e.g., GD2 or GD3). In some embodiments, a monoclonal antibody, or antigen-binding fragment thereof, specifically binds a carbohydrate portion of a ganglioside (e.g., GD2 or GD3).

In certain aspects, an antibody for use in the methods of the present disclosure is a conjugate comprising the monoclonal antibody, or antigen-binding fragment thereof, described herein is provided. In some such embodiments, the conjugate comprises an additional moiety such as, for example and without limitation, a second therapeutic agent, a detectable moiety (e.g., a fluorophore, an enzyme, a radioisotope, etc.), and the like.

In certain aspects, an antibody for use in the methods of the present disclosure comprises an immunoglobulin heavy and/or light chain selected from the group consisting of immunoglobulin heavy and light chain sequences listed in Table 2.

### Sequence Identity/Homology

Function-conservative variants are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A function-conservative variant also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

The percent identity between two sequences is a function of the number of identical positions shared by the sequences (i.e., % identity=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available on the world wide web at the GCG company website), using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11 17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444 453 (1970)) algorithm which has been incorporated into the GAP program in the GCG software package (available on the world wide web at the GCG company website), using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences for use in the methods of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403 10. BLAST nucleotide searches can be performed with the NBLAST program, score=100, wordlength=12 to obtain nucleotide sequences homologous to the nucleic acid molecules of the present disclosure. BLAST protein searches can be performed with the XBLAST program, score=50, wordlength=3 to obtain amino acid sequences homologous to the protein molecules of the present disclosure. To obtain gapped alignments for comparison purposes, Gapped BLAST can be utilized as described in Altschul et al., (1997) Nucleic Acids Res. 25(17):3389 3402. When utilizing BLAST and Gapped BLAST programs, the default parameters of the respective programs (e.g., XBLAST and NBLAST) can be used (available on the world wide web at the NCBI website).

### Sequences

As used herein, coding region refers to regions of a nucleotide sequence comprising codons which are translated into amino acid residues, whereas noncoding region refers to regions of a nucleotide sequence that are not translated into amino acids (e.g., 5′ and 3′ untranslated regions).

Complement [to] or complementary refers to the broad concept of sequence complementarity between regions of two nucleic acid strands or between two regions of the same nucleic acid strand. It is known that an adenine residue of a first nucleic acid region is capable of forming specific hydrogen bonds (base pairing) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least or about 50%, and preferably at least or about 75%, at least or about 90%, or at least or about 95% of the nucleotide residues of the first portion are capable of base pairing with nucleotide residues in the second portion. In other embodiments, all nucleotide residues of the first portion are capable of base pairing with nucleotide residues in the second portion.

A nucleic acid is operably linked when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, |
| | CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, |
| | TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, |
| | TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

In view of the foregoing, the nucleotide sequence of a DNA or RNA can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

TABLE 1
Exemplary sequences of the CDRs of anti-ganglioside monoclonal antibodies
for use in the methods of the present disclosure.

| Clone | | CDR | | Amino Acid |
|---|---|---|---|---|
| No. | Binds: | No. | cDNA Sequence | Sequence |
| 4 | GD2/GD3 | HC- | GGCTACACATTTACCAGGTACTGG | GYTFTRYW |
| Mouse | | CDR1 | (SEQ ID NO: 1) | (SEQ ID NO: 2) |
| mAb | | HC- | ATTTATCCTGGAAATAGTGATACT | IYPGNSDT |
| | | CDR2 | (SEQ ID NO: 3) | (SEQ ID NO: 4) |
| | | HC- | GCAAGATCCGATGGTCCTATGGACTAC | ARSDGPMDY |
| | | CDR3 | (SEQ ID NO: 5) | (SEQ ID NO: 6) |
| | | LC- | GAAAGTGTTGATAATTATGGCATCAGTT | ESVDNYGISF |
| | | CDR1 | TT | (SEQ ID NO: 8) |
| | | | (SEQ ID NO: 7) | |
| | | LC- | GCTGCATCC | AAS |
| | | CDR2 | (SEQ ID NO: 9) | (SEQ ID NO: 10) |
| | | LC- | CAGCAAAGTAAGGAGGTTCCGTTCACG | QQSKEVPFT |
| | | CDR3 | (SEQ ID NO: 11) | (SEQ ID NO: 12) |
| 6 | GD3 | HC- | ACCTATGGAATGAGC | TYGMS |
| Mouse | | CDR1 | (SEQ ID NO: 13) | (SEQ ID NO: 14) |
| IgG | | HC- | TGGATAAACACATATACTGGAGTGCCAA | WINTYTGVPTYGDDFKG |
| Kappa | | CDR2 | CATATGGTGATGACTTCAAGGGA | (SEQ ID NO: 16) |
| | | | (SEQ ID NO: 15) | |
| | | HC- | TGGTTACGCCACCATGCTATGGACTAC | WLRHHAMDY |
| | | CDR3 | (SEQ ID NO: 17) | (SEQ ID NO: 18) |
| | | LC- | AAGGCCAGTGAGAATGTGGTTACTTATG | KASENVVTYVS |
| | | CDR1 | TTTCC | (SEQ ID NO: 20) |
| | | | (SEQ ID NO: 19) | |
| | | LC- | GGGGCATCCAACCGGTACACT | GASNRYT |
| | | CDR2 | (SEQ ID NO: 21) | (SEQ ID NO: 22) |
| | | LC- | GGACAGGGTTACAGCTATCCGTACACG | GQGYSYPYT |
| | | CDR3 | (SEQ ID NO: 23) | (SEQ ID NO: 24) |
| 7 | GD2/GD3 | HC- | GGATTCACTTTTAGTGACGCCTGG | GFTFSDAW |
| Mouse | | CDR1 | (SEQ ID NO: 25) | (SEQ ID NO: 26) |
| mAb | | HC- | ATTAGAAACAAAGCTAATAATCATGCG | IRNKANNHAT |
| | | CDR2 | ACA | (SEQ ID NO: 28) |
| | | | (SEQ ID NO: 27) | |
| | | HC- | ACCAGGCGACATGATTCCTACTTTGACT | TRRHDSYFDY |
| | | CDR3 | AC | (SEQ ID NO: 30) |
| | | | (SEQ ID NO: 29) | |
| | | LC- | CAGGATGTGGATACTGCT | QDVDTA |
| | | CDR1 | (SEQ ID NO: 31) | (SEQ ID NO: 32) |
| | | LC- | TGGGCATCC | WAS |
| | | CDR2 | (SEQ ID NO: 33) | (SEQ ID NO: 34) |
| | | LC- | CAGCAATATCGCAGCTATCCTCTCACG | QQYRSYPLT |
| | | CDR3 | (SEQ ID NO: 35) | (SEQ ID NO: 36) |
| 8 | GD2/GD3 | HC- | GGCTACACATTTACCAGTTACTGG | GYTFTSYW |
| Mouse | | CDR1 | (SEQ ID NO: 37) | (SEQ ID NO: 38) |
| mAb | | HC- | ATTTATCCTGGAAAAAGTGGTACT | IYPGKSGT |
| | | CDR2 | (SEQ ID NO: 39) | (SEQ ID NO: 40) |
| | | HC- | ACAAGATCCGATGGTCCTATGGACTAC | TRSDGPMDY |
| | | CDR3 | (SEQ ID NO: 41) | (SEQ ID NO: 42) |
| | | LC- | GAGAGTGTTGATAATTATGACATTAGTT | ESVDNYDISF |
| | | CDR1 | TT | (SEQ ID NO: 44) |
| | | | (SEQ ID NO: 43) | |
| | | LC- | GCTGCATCC | AAS |
| | | CDR2 | (SEQ ID NO: 45) | (SEQ ID NO: 46) |
| | | LC- | CAGCAAAGTAAGGAGGTTCCGTACACG | QQSKEVPYT |
| | | CDR3 | (SEQ ID NO: 47) | (SEQ ID NO: 48) |
| 9 | GD2/GD3 | HC- | GACTACAACATGGAC | DYNMD |
| Mouse | | CDR1 | (SEQ ID NO: 49) | (SEQ ID NO: 50) |
| IgG | | HC- | GATATTAATCCTAACAATGGTGGTACTA | DINPNNGGTIYNQKFKG |
| Kappa | | CDR2 | TCTACAACCAGAAGTTCAAGGGC | (SEQ ID NO: 52) |
| | | | (SEQ ID NO: 51) | |
| | | HC- | TCGGGGATCTACTATGATTACGCCTGGT | SGIYYDYAWFPY |
| | | CDR3 | TTCCTTAC | (SEQ ID NO: 54) |
| | | | (SEQ ID NO: 53) | |
| | | LC- | AGTGCAAGTCAGGGCATTAGCAATTATT | SASQGISNYLN |
| | | CDR1 | TAAAC | (SEQ ID NO: 56) |
| | | | (SEQ ID NO: 55) | |
| | | LC- | TACACATCAAGTTTACACTCA | YTSSLHS |
| | | CDR2 | (SEQ ID NO: 57) | (SEQ ID NO: 58) |
| | | LC- | CAGCAGTATAGTAAGCTTCCTCCTACG | QQYSKLPPT |
| | | CDR3 | (SEQ ID NO: 59) | (SEQ ID NO: 60) |
| 10 | GD2/GD3 | HC- | GACTACAACATGGAC | DYNMD |
| Mouse | | CDR1 | (SEQ ID NO: 61) | (SEQ ID NO: 62) |
| IgM | | HC- | GATATTAATCCTAACAATGGTGGTACTA | DINPNNGGTIYNQKFKG |
| Kappa | | CDR2 | TCTACAACCAGAAGTTCAAGGGC | (SEQ ID NO: 64) |
| | | | (SEQ ID NO: 63) | |
| | | HC- | TCGGGGATCTACTATGATTACGCCTGGT | SGIYYDYAWFPY |
| | | CDR3 | TTCCTTAC | (SEQ ID NO: 66) |
| | | | (SEQ ID NO: 65) | |
| | | LC- | AGTGCAAGTCAGGGCATTAGCAATTATT | SASQGISNYLN |
| | | CDR1 | TAAAC | (SEQ ID NO: 68) |
| | | | (SEQ ID NO: 67) | |
| | | LC- | TACACATCAAGTTTACACTCA | YTSSLHS |
| | | CDR2 | (SEQ ID NO: 69) | (SEQ ID NO: 70) |
| | | LC- | CAGCAGTATAGTAAGCTTCCTCCTACG | QQYSKLPPT |
| | | CDR3 | (SEQ ID NO: 71) | (SEQ ID NO: 72) |
| 13 | GD2/GD3 | HC- | GACTATGAAATGCAC | DYEMH |
| Mouse | | CDR1 | (SEQ ID NO: 73) | (SEQ ID NO: 74) |
| IgM | | HC- | GCTATTGATCCTGAAACTGGTGGTACTG | AIDPETGGTAYNQKFKG |
| Kappa | | CDR2 | CCTACAATCAGAAGTTCAAGGGC | (SEQ ID NO: 76) |
| | | | (SEQ ID NO: 75) | |
| | | HC- | AGCTGGGACGGAGACTAC | SWDGDY |
| | | CDR3 | (SEQ ID NO: 77) | (SEQ ID NO: 78) |
| | | LC- | AAGGCCAGTCAGAATGTGGGTACTAAT | KASQNVGTNVA |
| | | CDR1 | GTAGCC | (SEQ ID NO: 80) |
| | | | (SEQ ID NO: 79) | |
| | | LC- | TCGGCATCCTACCGGTACAGT | SASYRYS |
| | | CDR2 | (SEQ ID NO: 81) | (SEQ ID NO: 82) |
| | | LC- | CAGCAATATAACAGCTATCCATTCACG | QQYNSYPFT |
| | | CDR3 | (SEQ ID NO: 83) | (SEQ ID NO: 84) |
| 14 | GD2/GD3 | HC- | GACTACAACATGGAC | DYNMD |
| Mouse | | CDR1 | (SEQ ID NO: 85) | (SEQ ID NO: 86) |
| IgM | | HC- | GATATTAATCCTAACAATGGTGGTACTA | DINPNNGGTIYNQKFKG |
| Kappa | | CDR2 | TCTACAACCAGAAGTTCAAGGGC | (SEQ ID NO: 88) |
| | | | (SEQ ID NO: 87) | |
| | | HC- | TCGGGGATCTACTATGATTACGCCTGGT | SGIYYDYAWFPY |
| | | CDR3 | TTCCTTAC | (SEQ ID NO: 90) |
| | | | (SEQ ID NO: 89) | |
| | | LC- | AGTGCAAGTCAGGGCATTAGCAATTATT | SASQGISNYLN |
| | | CDR1 | TAAAC | (SEQ ID NO: 92) |
| | | | (SEQ ID NO: 91) | |
| | | LC- | TACACATCAAGTTTACACTCA | YTSSLHS |
| | | CDR2 | (SEQ ID NO: 93) | (SEQ ID NO: 94) |
| | | LC- | CAGCAGTATAGTAAGCTTCCTCCTACG | QQYSKLPPT |
| | | CDR3 | (SEQ ID NO: 95) | (SEQ ID NO: 96) |
| 15 | GD2/GD3 | HC- | GGATTCACTTTTAGTGACGCCTGG | GFTFSDAW |
| Mouse | | CDR1 | (SEQ ID NO: 97) | (SEQ ID NO: 98) |
| mAb | | HC- | ATTAGAAACAAAGCTAATAATCATGCG | IRNKANNHAT |
| | | CDR2 | ACA | (SEQ ID NO: 100) |
| | | | (SEQ ID NO: 99) | |
| | | HC- | ACCGGGCGACATGATTCCTACTTTGACT | TGRHDSYFDY |
| | | CDR3 | AC | (SEQ ID NO: 102) |
| | | | (SEQ ID NO: 101) | |
| | | LC- | CAGGGCATTAGCAATTAT | QGISNY |
| | | CDR1 | (SEQ ID NO: 103) | (SEQ ID NO: 104) |
| | | LC- | TACACATCA | YTS |
| | | CDR2 | (SEQ ID NO: 105) | (SEQ ID NO: 106) |
| | | LC- | CAGCAGTATAGTAAGCTTCCTCCTACG | QQYSKLPPT |
| | | CDR3 | (SEQ ID NO: 107) | (SEQ ID NO: 108) |
| 17 | GD2/GD3 | HC- | GGCTACACCTTCACCAGCTACTGG | GYTFTSYW |
| Mouse | | CDR1 | (SEQ ID NO: 109) | (SEQ ID NO: 110) |
| mAb | | HC- | ATTTATCCTGGTAGTGGTAGTACT | IYPGSGST |
| | | CDR2 | (SEQ ID NO: 111) | (SEQ ID NO: 112) |
| | | HC- | GCAAGCCACCGATTTGATTACTACGGTA | ASHRFDYYGSSYYAMDY |
| | | CDR3 | GTAGCTACTATGCTATGGACTAC | (SEQ ID NO: 114) |
| | | | (SEQ ID NO: 113) | |
| | | LC- | CAGGACATTTGCAATTAT | QDICNY |
| | | CDR1 | (SEQ ID NO: 115) | (SEQ ID NO: 116) |
| | | LC- | TACACATCA | YTS |
| | | CDR2 | (SEQ ID NO: 117) | (SEQ ID NO: 118) |
| | | LC- | CAACAGGGTAATACGCTTCCGCTCACG | QQGNTLPLT |
| | | CDR3 | (SEQ ID NO: 119) | (SEQ ID NO: 120) |
| 18 | GD2/GD3 | HC- | GGATACACGTTCACTGACTTCCAC | GYTFTDFH |
| Mouse | | CDR1 | (SEQ ID NO: 121) | (SEQ ID NO: 122) |
| mAb | | HC- | ATTAATCCTAACAATGGTGGTACT | INPNNGGT |
| | | CDR2 | (SEQ ID NO: 123) | (SEQ ID NO: 124) |
| | | HC- | GTAAGAGAAATCTACTTTGGCTTTGACT | VREIYFGFDY |
| | | CDR3 | AC | (SEQ ID NO: 126) |
| | | | (SEQ ID NO: 125) | |
| | | LC- | CAGGACATTTGCAATTAT | QDICNY |
| | | CDR1 | (SEQ ID NO: 127) | (SEQ ID NO: 128) |
| | | LC- | TACACATCA | YTS |
| | | CDR2 | (SEQ ID NO: 129) | (SEQ ID NO: 130) |
| | | LC- | CAACAGGGTAATACGCTTCCGCTCACG | QQGNTLPLT |
| | | CDR3 | (SEQ ID NO: 131) | (SEQ ID NO: 132) |
| 19 | GD2 | HC- | GGATACACATTCACTAAATACACC | GYTFTKYT |
| Mouse | | CDR1 | (SEQ ID NO: 133) | (SEQ ID NO: 134) |
| mAb | | HC- | ATTAATCCTAACAATGGTGGTACT | INPNNGGT |
| | | CDR2 | (SEQ ID NO: 135) | (SEQ ID NO: 136) |
| | | HC- | ACAAGCAAGTCCTTTGACTAC | TSKSFDY |
| | | CDR3 | (SEQ ID NO: 137) | (SEQ ID NO: 138) |
| | | LC- | TCAAGTGTAAGTAAC | SSVSN |
| | | CDR1 | (SEQ ID NO: 139) | (SEQ ID NO: 140) |
| | | LC- | AGCACATCC | STS |
| | | CDR2 | (SEQ ID NO: 141) | (SEQ ID NO: 142) |
| | | LC- | CAACAAAGGAGTGGTTACCCATTCACG | QQRSGYPFT |
| | | CDR3 | (SEQ ID NO: 143) | (SEQ ID NO: 144) |

TABLE 2
Exemplary sequences of the leader and variable regions of anti-ganglioside
monoclonal antibodies for use in the methods of the present disclosure.

SEQ ID NO: 145 Clone 4 Heavy Chain Variable (vH) cDNA Sequence
CAAGTACAGCTGGAGGAGTCTGGGACTGTGCTGGCAAGGCCTGGGGCTTCAGTGAAGATGTCCTGCAAGACTT
CTGGCTACACATTTACCAGGTACTGGATGCACTGGGTAAAACAGAGGCCTGGACAGGGTCTGGAATGGATAGG
GGCTATTTATCCTGGAAATAGTGATACTACCTACAACCAGAAGTTCAAGGGCAAGGCCAAACTGACTGCAGTC
ACATCCGCCACCAATGCCTACATGGAAGTAAGCAGCCTGACAAATGAGGACTCTGCGGTCTATTACTGTGCAA
GATCCGATGGTCCTATGGACTACTGGGGTCAAGGAACCTCAGTCACCGTCTCCTCA
SEQ ID NO: 146 Clone 4 Heavy Chain Variable (vH) Amino Acid Sequence
QVQLEESGTVLARPGASVKMSCKTSGYTFTRYWMHWVKQRPGQGLEWIGAIYPGNSDTTYNQKFKGKAKLTAV
TSATNAYMEVSSLTNEDSAVYYCARSDGPMDYWGQGTSVTVSS
SEQ ID NO: 147 Clone 4 Light Chain (kappa) Variable (vL) cDNA Sequence
GACATTGTACTGACCCAATCTCCAGCTTCTTTGGCTGTGTCTCTAGGGCAGAGGGCCACCATTTCCTGCAGAG
CCAGCGAAAGTGTTGATAATTATGGCATCAGTTTTATGAACTGGTTCCAACAGAAACCAGGACAGCCACCCAA
ACTCCTCATCTATGCTGCATCCAATCAAGGATCCGGGGTCCCTGCCAGGTTTAGTGGCAGTGGGTCTGGGACA
GACTTCAGTCTCAACATCCATCCTATGGAGGAGGATGATACTGCAATGTATTTCTGTCAGCAAAGTAAGGAGG
TTCCGTTCACGTTCGGAGGGGGGACCAAGCTGGA
SEQ ID NO: 148 Clone 4 Light Chain (kappa) Variable (vL) Amino Acid
Sequence
DIVLTQSPASLAVSLGQRATISSCRASESVDNYGISFMNWFQQKPGQPPKLLIYAASNQGSGVPARFSGSGSGT
DFSLNIHPMEEDDTAMYFCQQSKEVPFTFGGGTKL
SEQ ID NO: 149 Clone 6 Heavy Chain Variable (vH) cDNA Sequence
CAGATCCAGTTGGTACAATCTGGACCTGAGCTGAAGAAGCCTGGAGAGACAGTCAAGATCTCCTGCAAGGCTT
CTGGATATTCCTTCACAACCTATGGAATGAGCTGGGTGAAACAGGCTCCAGGAAAGGGTTTAAAGTGGATGGG
CTGGATAAACACATATACTGGAGTGCCAACATATGGTGATGACTTCAAGGGACGGTTTGCCTTCTCTTTGGAA
ACCTCTACCAGCACTGCCTATTTGCAGATCAACAACCTCAAAAATGACGACACGGCTTCATATTTCTGTGCAA
GATGGTTACGCCACCATGCTATGGACTACTGGGGTCAAGGAACCTCAGTCACCGTCTCCTCA
SEQ ID NO: 150 Clone 6 Heavy Chain Variable (vH) Amino Acid Sequence
QIQLVQSGPELKKPGETVKISCKASGYSFTTYGMSWVKQAPGKGLKWM
GWINTYTGVPTYGDDFKGRFAFSLETSTSTAYLQINNLKNDDTASYFCARWLRHHAMDYWGQGTSVTVSS
SEQ ID NO: 151 Clone 6 Light Chain (kappa) Variable (vL) cDNA Sequence
AACATTGTAATGACCCAATCTCCCAAATCCATGTCCATGTCAGTAGGAGAGAGGGTCACCTTGACCTGCAAGG
CCAGTGAGAATGTGGTTACTTATGTTTCCTGGTATCAACAGAAACCAGAGCAGTCTCCTAAACTGCTGATATA
CGGGGCATCCAACCGGTACACTGGGGTCCCCGATCGCTTCACAGGCAGTGGATCTGCAACAGATTTCACTCTG
ACCATCAGCAGTGTGCAGGCTGAAGACCTTGCAGATTATCACTGTGGACAGGGTTACAGCTATCCGTACACGT
TCGGAGGGGGGACCAAGCTGGAAATAAAA
SEQ ID NO: 152 Clone 6 Light Chain (kappa) Variable (vL) Amino Acid
Sequence
NIVMTQSPKSMSMSVGERVTLTCKASENVVTYVSWYQQKPEQSPKLLIYGASNRYTGVPDRFTGSGSATDFTL
TISSVQAEDLADYHCGQGYSYPYTFGGGTKLEIK
SEQ ID NO: 153 Clone 7 Heavy Chain Variable (vH) cDNA Sequence
CAGGTTCAGCTGGAGCAGTCTGGAGGAGCCTTGGTGCAACCTGGAGGATCCATGAAACTCTCTTGTGCTGCCT
CTGGATTCACTTTTAGTGACGCCTGGATGGACTGGGTCCGCCAGTCTCCAGAAAAGGGGCTTGAGTGGGTTGC
TGAAATTAGAAACAAAGCTAATAATCATGCGACATACTATGCTGAGTCTGTGAAAGGGAGGTTCACCATCTCA
AGAGATGATTCCAAAAATAGTGTCTACCTGCAAATGAACAACTTAAGAGCTGAAGACACTGGCATTTATTATT
GTACCAGGCGACATGATTCCTACTTTGACTACTGGGGCCAAGGCACCACTCTCACAGTCTCCTCA
SEQ ID NO: 154 Clone 7 Heavy Chain Variable (vH) Amino Acid Sequence
QVQLEQSGGALVQPGGSMKLSCAASGFTFSDAWMDWVRQSPEKGLEWVAEIRNKANNHATYYAESVKGRFTIS
RDDSKNSVYLQMNNLRAEDTGIYYCTRRHDSYFDYWGOGTTLTVSS
SEQ ID NO: 155 Clone 7 Light Chain (kappa) Variable (vL) cDNA Sequence
GACATTGTGATGACCCAGTCTCACAAATTCATGTCCACATCAGTAGGAGACAGGGTCAGCATCACCTACAAGG
CCAGTCAGGATGTGGATACTGCTGTAGCCTGGTATCAACAGAAACCAGGGCAATCTCCTAAACTACTGATTTA
CTGGGCATCCACCCGGCACACTGGAGTCCCTGATCGCTTCACAGGCAGTGGATCTGGGACAGATTTCACTCTC
ACCATTAACAATGTGCAGTCTGAAGACTTGGCAGATTATTTCTGTCAGCAATATCGCAGCTATCCTCTCACGT
TCGGTGCTGGGACCAAGCTGGAACTGAAACGG
SEQ ID NO: 156 Clone 7 Light Chain (kappa) Variable (vL) Amino Acid
Sequence
DIVMTQSHKFMSTSVGDRVSITYKASQDVDTAVAWYQQKPGQSPKLLIYWASTRHTGVPDRFTGSGSGTDFTL
TINNVQSEDLADYFCQQYRSYPLTFGAGTKLELKR
SEQ ID NO: 157 Clone 8 Heavy Chain Variable (vH) cDNA Sequence
GAAGTAAAGCTGCAGGAGTCTGGGACTGAGCTGGCAAGGCCTGGGGCTTCAGTGAAGATGTCCTGCAAGACTT
CTGGCTACACATTTACCAGTTACTGGATGCACTGGGTAAAACAGAGGCCTGGACAGGGTCTGGAATGGATAGG
GGCTATTTATCCTGGAAAAAGTGGTACTACCTACAACCAGAAGTTCAAGGGCAAGGCCAAACTGACTGCAGTC
ACATCCGCCAGCACTGCCTACATGGAACTCAGCAGCCTGACAAATGAGGACTCTGCGGTCTATTACTGTACAA
GATCCGATGGTCCTATGGACTACTGGGGTCAAGGAACCTCAGTCACCGTCTCCTCA
SEQ ID NO: 158 Clone 8 Heavy Chain Variable (vH) Amino Acid Sequence
EVKLQESGTELARPGASVKMSCKTSGYTFTSYWMHWVKQRPGQGLEWIGAIYPGKSGTTYNQKFKGKAKLTAV
TSASTAYMELSSLTNEDSAVYYCTRSDGPMDYWGQGTSVTVSS
SEQ ID NO: 159 Clone 8 Light Chain (kappa) Variable (vL) cDNA Sequence
GACATTGTGCTGACCCAATCTCCAGCTTCTTTGGCTGTGTCTCTAGGGCAGAGGGCCACCATCTCCTGCAGAG
CCAGCGAGAGTGTTGATAATTATGACATTAGTTTTATGAACTGGTTCCAACAGAAACCAGGACAGCCACCCAA
ACTCCTCATCTATGCTGCATCCAACCAAGGATCCGGGGTCCCTGCCAGGTTCAGTGGCAGTGGGTCTGGGACA
GACTTCAGTCTCAACATCCATCCTATGGAGGAGGATGATACTGCAATGTATTTCTGTCAGCAAAGTAAGGAGG
TTCCGTACACGTTCGGAGGGGGGACCAAGCTGGAAATAAAACGG
SEQ ID NO: 160 Clone 8 Light Chain (kappa) Variable (vL) Amino Acid
Sequence
DIVLTQSPASLAVSLGQRATISCRASESVDNYDISFMNWFQQKPGQPPKLLIYAASNQGSGVPARFSGSGSGT
DFSLNIHPMEEDDTAMYFCQQSKEVPYTFGGGTKLEIKR
SEQ ID NO: 161 Clone 9 Heavy Chain Variable (vH) cDNA Sequence
GAGGTCCAGCTGCAACAGTCTGGACCTGAGCTGGTGAAGCCTGGGGCTTCAGTGAAGATACCCTGCAAGGCTT
CTGGATACACATTCACTGACTACAACATGGACTGGGTGAAACAGAGCCATGGAAAGAGCCTTGAGTGGATTGG
AGATATTAATCCTAACAATGGTGGTACTATCTACAACCAGAAGTTCAAGGGCAAGGCCACATTGACTGTAGAC
AAGTCCTCCAGCACAGCCTACATGGAGCTCCGCAGCCTGACATCTGAGGACACTGCAGTCTATTACTGTGCAA
GATCGGGGATCTACTATGATTACGCCTGGTTTCCTTACTGGGGCCAAGGGACTCTGGTCACTGTCTCTGCA
SEQ ID NO: 162 Clone 9 Heavy Chain Variable (vH) Amino Acid Sequence
EVQLQQSGPELVKPGASVKIPCKASGYTFTDYNMDWVKQSHGKSLEWIGDINPNNGGTIYNQKFKGKATLTVD
KSSSTAYMELRSLTSEDTAVYYCARSGIYYDYAWFPYWGQGTLVTVSA
SEQ ID NO: 163 Clone 9 Light Chain (kappa) Variable (vL) cDNA Sequence
GATATCCAGATGACACAGACTACATCCTCCCTGTCTGCCTCTCTGGGAGACAGAGTCACCATCAGTTGCAGTG
CAAGTCAGGGCATTAGCAATTATTTAAACTGGTATCAGCAGAAACCAGATGGAACTGTTAAACTCCTGATCTA
TTACACATCAAGTTTACACTCAGGAGTCCCATCAAGGTTCAGTGGCAGTGGGTCTGGGACAGATTATTCTCTC
ACCATCAGCAACCTGGAACCTGAAGATATTGCCACTTACTATTGTCAGCAGTATAGTAAGCTTCCTCCTACGT
TCGGTGCTGGGACCAAGCTGGAGCTGAAA
SEQ ID NO: 164 Clone 9 Light Chain (kappa) Variable (vL) Amino Acid
Sequence
DIQMTQTTSSLSASLGDRVTISCSASQGISNYLNWYQQKPDGTVKLLIYYTSSLHSGVPSRFSGSGSGTDYSL
TISNLEPEDIATYYCQQYSKLPPTFGAGTKLELK
SEQ ID NO: 165 Clone 10 Heavy Chain Variable (vH) cDNA Sequence
GAGGTCCAGCTGCAACAGTCTGGACCTGAGCTGGTGAAGCCTGGGGCTTCAGTGAAGATACCCTGCAAGGCTT
CTGGATACACATTCACTGACTACAACATGGACTGGGTGAAACAGAGCCATGGAAAGAGCCTTGAGTGGATTGG
AGATATTAATCCTAACAATGGTGGTACTATCTACAACCAGAAGTTCAAGGGCAAGGCCACATTGACTGTAGAC
AAGTCCTCCAGCACAGCCTACATGGAGCTCCGCAGCCTGACATCTGAGGACACTGCAGTCTATTACTGTGCAA
GATCGGGGATCTACTATGATTACGCCTGGTTTCCTTACTGGGGCCAAGGGACTCTGGTCACTGTCTCTGCAG
SEQ ID NO: 166 Clone 10 Heavy Chain Variable (vH) Amino Acid Sequence
EVQLQQSGPELVKPGASVKIPCKASGYTFTDYNMDWVKQSHGKSLEWIGDINPNNGGTIYNQKFKGKATLTVD
KSSSTAYMELRSLTSEDTAVYYCARSGIYYDYAWFPYWGQGTLVTVSA
SEQ ID NO: 167 Clone 10 Light Chain (kappa) Variable (vL) cDNA Sequence
GATATCCAGATGACACAGACTACATCCTCCCTGTCTGCCTCTCTGGGAGACAGAGTCACCATCAGTTGCAGTG
CAAGTCAGGGCATTAGCAATTATTTAAACTGGTATCAGCAGAAACCAGATGGAACTGTTAAACTCCTGATCTA
TTACACATCAAGTTTACACTCAGGAGTCCCATCAAGGTTCAGTGGCAGTGGGTCTGGGACAGATTATTCTCTC
ACCATCAGCAACCTGGAACCTGAAGATATTGCCACTTACTATTGTCAGCAGTATAGTAAGCTTCCTCCTACGT
TCGGTGCTGGGACCAAGCTGGAGCTGAAAC
SEQ ID NO: 168 Clone 10 Light Chain (kappa) Variable (vL) Amino Acid
Sequence
DIQMTQTTSSLSASLGDRVTISCSASQGISNYLNWYQQKPDGTVKLLIYYTSSLHSGVPSRFSGSGSGTDYSL
TISNLEPEDIATYYCQQYSKLPPTFGAGTKLELK
SEQ ID NO: 169 Clone 13 Heavy Chain Variable (vH) cDNA Sequence
CAGGTTCAACTGCAGCAGTCTGGGGCTGAGCTGGTGAGGCCTGGGGCTTCAGTGACGCTGTCCTGCAAGGCTT
CGGGCTACACATTTACTGACTATGAAATGCACTGGGTGAAGCAGACACCTGTGCATGGCCTGGAATGGATTGG
AGCTATTGATCCTGAAACTGGTGGTACTGCCTACAATCAGAAGTTCAAGGGCAAGGCCATACTGACTGCAGAC
AAATCCTCCAGCACAGCCTACATGGAGCTCCGCAGCCTGACATCTGAGGACTCTGCCGTCTATTACTGTACAA
GAAGCTGGGACGGAGACTACTGGGGCCAAGGCACCACTCTCACAGTCTCCTCA
SEQ ID NO: 170 Clone 13 Heavy Chain Variable (vH) Amino Acid Sequence
QVQLQQSGAELVRPGASVTLSCKASGYTFTDYEMHWVKQTPVHGLEWIGAIDPETGGTAYNQKFKGKAILTAD
KSSSTAYMELRSLTSEDSAVYYCTRSWDGDYWGOGTTLTVSS
SEQ ID NO: 171 Clone 13 Light Chain (kappa) Variable (vL) cDNA Sequence
GACATTGTGATGACCCAGTCTCAAAAATTCATGTCCACATCAGTAGGAGACAGGGTCAGCGTCACCTGCAAGG
CCAGTCAGAATGTGGGTACTAATGTAGCCTGGTATCAACAGAAACCAGGGCAATCTCCTAAAGCACTGATTTA
CTCGGCATCCTACCGGTACAGTGGAGTCCCTGATCGCTTCACAGGCAGTGGATCTGGGACAGATTTCACTCTC
ACCATCAGCAATGTGCAGTCTGAAGACTTGGCAGAGTATTTCTGTCAGCAATATAACAGCTATCCATTCACGT
TCGGCTCGGGGACAAAGTTGGAAATAAAA
SEQ ID NO: 172 Clone 13 Light Chain (kappa) Variable (vL) Amino Acid
Sequence
DIVMTQSQKFMSTSVGDRVSVTCKASQNVGTNVAWYQQKPGQSPKALIYSASYRYSGVPDRFTGSGSGTDFTL
TISNVQSEDLAEYFCQQYNSYPFTFGSGTKLEIK
SEQ ID NO: 173 Clone 14 Heavy Chain Variable (vH) cDNA Sequence
GAGGTCCAGCTGCAACAGTCTGGACCTGAGCTGGTGAAGCCTGGGGCTTCAGTGAAGATACCCTGCAAGGCTT
CTGGATACACATTCACTGACTACAACATGGACTGGGTGAAACAGAGCCATGGAAAGAGCCTTGAGTGGATTGG
AGATATTAATCCTAACAATGGTGGTACTATCTACAACCAGAAGTTCAAGGGCAAGGCCACATTGACTGTAGAC
AAGTCCTCCAGCACAGCCTACATGGAGCTCCGCAGCCTGACATCTGAGGACACTGCAGTCTATTACTGTGCAA
GATCGGGGATCTACTATGATTACGCCTGGTTTCCTTACTGGGGCCAAGGGACTCTGGTCACTGTCTCTGCAG
SEQ ID NO: 174 Clone 14 Heavy Chain Variable (vH) Amino Acid Sequence
EVQLQQSGPELVKPGASVKIPCKASGYTFTDYNMDWVKQSHGKSLEWIGDINPNNGGTIYNQKFKGKATLTVD
KSSSTAYMELRSLTSEDTAVYYCARSGIYYDYAWFPYWGQGTLVTVSA
SEQ ID NO: 175 Clone 14 Light Chain (kappa) Variable (vL) cDNA Sequence
GATATCCAGATGACACAGACTACATCCTCCCTGTCTGCCTCTCTGGGAGACAGAGTCACCATCAGTTGCAGTG
CAAGTCAGGGCATTAGCAATTATTTAAACTGGTATCAGCAGAAACCAGATGGAACTGTTAAACTCCTGATCTA
TTACACATCAAGTTTACACTCAGGAGTCCCATCAAGGTTCAGTGGCAGTGGGTCTGGGACAGATTATTCTCTC
ACCATCAGCAACCTGGAACCTGAAGATATTGCCACTTACTATTGTCAGCAGTATAGTAAGCTTCCTCCTACGT
TCGGTGCTGGGACCAAGCTGGAGCTGAAAC
SEQ ID NO: 176 Clone 14 Light Chain (kappa) Variable (vL) Amino Acid
Sequence
DIQMTQTTSSLSASLGDRVTISCSASQGISNYLNWYQQKPDGTVKLLIYYTSSLHSGVPSRFSGSGSGTDYSL
TISNLEPEDIATYYCQQYSKLPPTFGAGTKLELK
SEQ ID NO: 177 Clone 15 Heavy Chain Variable (vH) cDNA Sequence
CAAGTTCAGCTGCAGGAGTCTGGAGGAGCCTTGGTGCAACCTGGAGGATCCATGAAACTCTCTTGTGCTGCCT
CTGGATTCACTTTTAGTGACGCCTGGATGGACTGGGTCCGCCAGTCTCCAGAAAAGGGGCTTGAGTGGGTTGC
TGAAATTAGAAACAAAGCTAATAATCATGCGACATACTATGCTGAGTCTGTGAAAGGGAGGTTCACCATCTCA
AGAGATGATTCCAAAAATAGTGTCTACCTGCAAATGAACAACTTAAGAGCTGAGGACACTGGCATTTATTACT
GTACCGGGCGACATGATTCCTACTTTGACTACTGGGGCCAAGGCACCACTCTCACAGTCTCCTCA
SEQ ID NO: 178 Clone 15 Heavy Chain Variable (vH) Amino Acid Sequence
QVQLQESGGALVQPGGSMKLSCAASGFTFSDAWMDWVRQSPEKGLEWVAEIRNKANNHATYYAESVKGRFTIS
RDDSKNSVYLQMNNLRAEDTGIYYCTGRHDSYFDYWGQGTTLTVSS
SEQ ID NO: 179 Clone 15 Light Chain (kappa) Variable (vL) cDNA Sequence
GATATCCAGATGACACAGACTACATCCTCCCTGTCTGCCTCTCTGGGAGACAGAGTCACCATCAGTTGCAGTG
CAAGTCAGGGCATTAGCAATTATTTAAACTGGTATCAGCAGAAACCAGATGGAACTGTTAAACTCCTGATCTA
TTACACATCAAGTTTACACTCAGGAGTCCCATCAAGGTTCAGTGGCAGTGGGTCTGGGACAGATTATTCTCTC
ACCATCAGCAACCTGGAACCTGAAGATATTGCCACTTACTATTGTCAGCAGTATAGTAAGCTTCCTCCTACGT
TCGGTGCTGGGACCAAGCTGGAGCTGAAACGG
SEQ ID NO: 180 Clone 15 Light Chain (kappa) Variable (vL) Amino Acid
Sequence
DIQMTQTTSSLSASLGDRVTISCSASQGISNYLNWYQQKPDGTVKLLIYYTSSLHSGVPSRFSGSGSGTDYSL
TISNLEPEDIATYYCQQYSKLPPTFGAGTKLELKR
SEQ ID NO: 181 Clone 17 Heavy Chain Variable (vH) cDNA Sequence
GAGGTCCAGCTGGAGGAGTCTGGGGCTGAGCTTGTGAAGCCTGGGGCTTCGGTGAAGATGTCCTGTAAGGCTT
CTGGCTACACCTTCACCAGCTACTGGATAACCTGGGTGAAGCAGAGGCCTGGACAAGGCCTTGAGTGGATTGG
AGATATTTATCCTGGTAGTGGTAGTACTAACTACAATGAGAAGTTCAAGAGCAAGGCCACACTGACTGTAGAC
ACATCCTCCAGCACAACCTACATGCAGCTCAGCAGCCTGACATCTGAGGACTCTGCGGTCTATTACTGTGCAA
GCCACCGATTTGATTACTACGGTAGTAGCTACTATGCTATGGACTACTGGGGTCAAGGAACCTCAGTCACCGT
CTCCTCA
SEQ ID NO: 182 Clone 17 Heavy Chain Variable (vH) Amino Acid Sequence
EVQLEESGAELVKPGASVKMSCKASGYTFTSYWITWVKQRPGOGLEWIGDIYPGSGSTNYNEKFKSKATLTVD
TSSSTTYMQLSSLTSEDSAVYYCASHRFDYYGSSYYAMDYWGQGTSVTVSS
SEQ ID NO: 183 Clone 17 Light Chain (kappa) Variable (vL) cDNA Sequence
GATATCCAGATGACACAGACTACATCCTCCCTGTCTGCCTCTCTGGGAGACAGAGTCACCATCAGTTGCAGGG
CAAGTCAGGACATTTGCAATTATTTAAACTGGTATCAGCAGAAACCAGATGGACCTTTTAAACTCCTGATCTT
CTACACATCAAGATTACACTCAGGAGTCCCATCAAGGTTCAGTGGCAGTGGGTCTGGAACAGATTATTCTCTC
ACCATTAGCAACCTGGAGCAAGAAGATATTGCCACTTACTTTTGCCAACAGGGTAATACGCTTCCGCTCACGT
TCGGTGCTGGGACCAAGCTGGAGCTGAAACGG
SEQ ID NO: 184 Clone 17 Light Chain (kappa) Variable (vL) Amino Acid
Sequence
DIQMTQTTSSLSASLGDRVTISCRASQDICNYLNWYQQKPDGPFKLLIFYTSRLHSGVPSRESGSGSGTDYSL
TISNLEQEDIATYFCQQGNTLPLTFGAGTKLELKR
SEQ ID NO: 185 Clone 18 Heavy Chain Variable (vH) cDNA Sequence
GAAGTACAGCTGGAGGAGTCTGGACCTGAGCTGGTGAAGCCTGGGACTTCAGTGAAGATATCCTGTAAGGCTT
CTGGATACACGTTCACTGACTTCCACATTAACTGGGTGAAACAGAGCCATGGAAAGAACCTTGAGTGGATTGG
AGATATTAATCCTAACAATGGTGGTACTAACTACAACCAGAAATTCAAGGGCAAGGCCACATTGATTGTTGAC
AAGTCTTCCAGCGCAGCCTACATGGAGCTCCGCAGCCTGACATCTGAGGACTCTGCAGTCTATTATTGTGTAA
GAGAAATCTACTTTGGCTTTGACTACTGGGGCCAAGGCACCACTCTCACAGTCTCCTCA
SEQ ID NO: 186 Clone 18 Heavy Chain Variable (vH) Amino Acid Sequence
EVQLEESGPELVKPGTSVKISCKASGYTFTDFHINWVKQSHGKNLEWIGDINPNNGGTNYNQKFKGKATLIVD
KSSSAAYMELRSLTSEDSAVYYCVREIYFGFDYWGQGTTLTVSS
SEQ ID NO: 187 Clone 18 Light Chain (kappa) Variable (vL) cDNA Sequence
GATATCCAGATGACACAGACTACATCCTCCCTGTCTGCCTCTCTGGGAGACAGAGTCACCATCAGTTGCAGGG
CAAGTCAGGACATTTGCAATTATTTAAACTGGTATCAGCAGAAACCAGATGGACCTTTTAAACTCCTGATCTT
CTACACATCAAGATTACACTCAGGAGTCCCATCAAGGTTCAGTGGCAGTGGGTCTGGAACAGATTATTCTCTC
ACCATTAGCAACCTGGAGCAAGAAGATATTGCCACTTACTTTTGCCAACAGGGTAATACGCTTCCGCTCACGT
TCGGTGCTGGGACCAAGCTGGAGCTGAAACGG
SEQ ID NO: 188 Clone 18 Light Chain (kappa) Variable (vL) Amino Acid
Sequence
DIQMTQTTSSLSASLGDRVTISCRASQDICNYLNWYQQKPDGPFKLLIFYTSRLHSGVPSRFSGSGSGTDYSL
TISNLEQEDIATYFCQQGNTLPLTFGAGTKLELKR
SEQ ID NO: 189 Clone 19 Heavy Chain Variable (vH) cDNA Sequence
GAAGTGAAGCTGGAGGAGTCTGGACCTGAGCTGGTGAAGCCTGGGGCTTCAGTGAAGATATCCTGCAAGACTT
CTGGATACACATTCACTAAATACACCATGCACTGGGTGAAGCAGAGCCATGGAAAGAGCCTTGAGTGGATTGG
AGATATTAATCCTAACAATGGTGGTACTAACTACAACCAGAAGTTCAAGGGCACGGCCACATTGACTGTACAC
AAGTCCTCCACCACAGCCTACATGGAGCTCCGCAGCCTGACATCTGAGGATTCTGCAGTCTATTACTGTACAA
GCAAGTCCTTTGACTACTGGGGCCAAGGCACCACTCTCACAGTCTCCTCA
SEQ ID NO: 190 Clone 19 Heavy Chain Variable (vH) Amino Acid Sequence
EVKLEESGPELVKPGASVKISCKTSGYTFTKYTMHWVKQSHGKSLEWIGDINPNNGGTNYNQKFKGTATLTVH
KSSTTAYMELRSLTSEDSAVYYCTSKSFDYWGQGTTLTVSS
SEQ ID NO: 191 Clone 19 Light Chain (kappa) Variable (vL) cDNA Sequence
CAAATTGTTCTCACCCAGTCTCCAGCAATCATGTCTGCATCTCCAGGGGAGAAGGTCACCATAACCTGCAGTG
CCAGCTCAAGTGTAAGTAACATACACTGGTTCCAGCAGAAGCCAGGCACTTTTCCCAAACTCTGGATTTATAG
CACATCCACCCTGGCTTCTGGAGTCCCTGGTCGCTTCAGTGGCAGTGGATCTGGGACCTCTTACTCTCTCACA
ATCAGCCGAATGGGGGCTGAAGATGCTGCCACTTATTACTGCCAACAAAGGAGTGGTTACCCATTCACGTTCG
GCTCGGGGACAAAGTTGGAAATAAAACGG
SEQ ID NO: 192 Clone 19 Light Chain (kappa) Variable (vL) Amino Acid
Sequence
QIVLTQSPAIMSASPGEKVTITCSASSSVSNIHWFQQKPGTFPKLWIY
STSTLASGVPGRFSGSGSGTSYSLTISRMGAEDAATYYCQQRSGYPFTFGSGTKLEIKR

* Included in Tables 1 and 2 are RNA nucleic acid molecules (e.g., thymidine replaced with uridine), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Tables 1 and 2, or a portion thereof. Such nucleic acid molecules can have a function of the full-length nucleic acid as described further herein.
*The antibodies presented in Tables 1 and 2 bind the carbohydrate domain of the ganglioside GD2 and GD3.

### Nucleic Acids, Vectors, and Recombinant Host Cells

A further object of the disclosure relates to nucleic acid sequences encoding monoclonal antibodies and fragments thereof, immunoglobulins, and polypeptides for use in the methods of the present disclosure.

For example, in certain embodiments, the present disclosure relates, in part, to a nucleic acid sequence encoding the vH domain or the vL domain of the antibodies or antigen-binding fragment thereof for use in the methods of the present disclosure.

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Thus, a vector comprising a nucleic acid may also be used in the methods of the present disclosure.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used. Examples of suitable vectors include pAGE107 (Miyaji H et al. 1990), pAGE103 (Mizukami T et al. 1987), pHSG274 (Brady Get al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

The nucleic acids described herein may be used to produce a recombinant polypeptide in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

In another aspect, the present disclosure provides isolated nucleic acids that hybridize under selective hybridization conditions to a polynucleotide disclosed herein for use in the methods of the present disclosure. Thus, the polynucleotides of this embodiment can be used for isolating, detecting, and/or quantifying nucleic acids comprising such polynucleotides. For example, polynucleotides can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. Preferably, the cDNA library comprises at least 80% full-length sequences, preferably, at least 85% or 90% full-length sequences, and, preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences. Optionally, polynucleotides will encode at least a portion of an antibody encoded by the polynucleotides described herein. The polynucleotides may embrace nucleic acid sequences that can be employed for selective hybridization to a polynucleotide encoding an antibody for use in the methods of the present disclosure. See, e.g., Ausubel, supra; Colligan, supra, each entirely incorporated herein by reference.

### Methods of Producing Antibodies

Antibodies and fragments thereof, immunoglobulins, and polypeptides for use in the methods of the present disclosure may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies and other polypeptides of the present disclosure can be synthesized by recombinant DNA techniques as is well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques. For example, an antibody or a polypeptide for use in the methods of the present disclosyre may be produced by a method comprising the steps of: (i) culturing a transformed host cell according to the disclosure under conditions suitable to allow expression of said antibody or polypeptide; and (ii) recovering the expressed antibody or polypeptide.

Antibodies and other polypeptides for use in the methods of the present disclosure may be suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Chimeric antibodies (e.g., mouse-human chimeras or non-rodent-human chimeras) can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody can be any region which belongs to human immunoglobulin, such as the IgG class or a subclass thereof, such as IgG1, IgG2, IgG3 and IgG4. Similarly, the CL of a human chimeric antibody can be any region which belongs to Ig, such as the kappa class or lambda class. chimeric and humanized monoclonal antibodies, comprising both human and non-human portions, which can be made using standard recombinant DNA techniques. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988)J Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. No. 5,565,332, or 5,733,743. Humanized antibodies for use in the methods of the present disclosure can be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell. The humanized antibody expression vector may be either of a type in which a gene encoding an antibody heavy chain and a gene encoding an antibody light chain exists on separate vectors or of a type in which both genes exist on the same vector (tandem type).

Methods for producing humanized antibodies based on conventional recombinant DNA and gene transfection techniques are well-known in the art (See, e.g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan EA (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

In addition, methods for producing antibody fragments are well-known. For example, Fab fragments of the present disclosure can be obtained by treating an antibody which specifically reacts with a ganglioside with a protease such as papain. Also, Fabs can be produced by inserting DNA encoding Fabs of the antibody into a vector for prokaryotic expression system, or for eukaryotic expression system, and introducing the vector into a procaryote or eucaryote (as appropriate) to express the Fabs.

Similarly, F(ab′)2 fragments of the present disclosure can be obtained treating an antibody which specifically reacts with a ganglioside with a protease, pepsin. Also, the F(ab′)2 fragment can be produced by binding Fab′ described below via a thioether bond or a disulfide bond.

Fab′ fragments of the present disclosure can be obtained treating F(ab′)2 which specifically reacts with a ganglioside with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present disclosure can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment, and grafting them onto a human scFv fragment framework of known three dimensional structure (see, e.g., WO98/45322; WO 87/02671; U.S. Pat. Nos. 5,859,205; 5,585,089; 4,816,567; EP0173494).

### Modification of Antibodies, Immunoglobulins, and Polypeptides

Amino acid sequence modification(s) of the antibodies described herein are contemplated for use in the methods of the present disclosure. For example, it may be desirable to improve the binding affinity and/or other biological properties of an antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Modifications and changes may be made in the structure of the antibodies for use in the methods of the present disclosure, and in the DNA sequences encoding them, and still obtain a functional molecule that encodes an antibody and polypeptide with desirable characteristics. For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the disclosure, or corresponding DNA sequences which encode said polypeptides, without appreciable loss of their biological activity.

In one embodiment, amino acid changes may be achieved by changing codons in the DNA sequence to encode conservative substitutions based on conservation of the genetic code. Specifically, there is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code (see genetic code chart above).

As described above, an important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

Another type of amino acid modification of the antibody of the disclosure may be useful for altering the original glycosylation pattern of the antibody to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins of the present disclosure with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6 diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

### Pharmaceutical Compositions

In some embodiments of methods of the present disclosure, an antibody, or antigen-binding fragment thereof, is administered in the form of a pharmaceutical composition comprising the antibody, or antigen-binding fragment thereof, and a pharmaceutically acceptable carrier. As used herein, the pharmaceutically acceptable carrier is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds can also be incorporated into the compositions.

A pharmaceutical composition for use in the methods of the present disclosure is formulated to be compatible with its intended route of administration. Examples of routes of administration include parenteral, e.g., intravenous, intradermal, subcutaneous, intraperitoneal, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition should be sterile and should be fluid to the extent that easy syringeability exists. It must be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Inhibition of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the compounds for use in the methods of the present disclosure are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations should be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present disclosure are dictated by, and directly dependent on, the unique characteristics of the active compound, the particular therapeutic effect to be achieved, and the limitations inherent in the art of compounding such an active compound for the treatment of individuals.

### Exemplary Embodiments of the Therapeutic Methods

In certain aspects, provided herein are therapeutic methods for the prevention and treatment of cancer. For example, in certain aspects, provided herein is a method of treating a cancer in a subject, the method comprising: administering to the subject a therapeutically effective amount of an antibody or antigen-binding fragment thereof, or pharmaceutical composition thereof, of the present disclosure, such that the cancer is treated in the subject.

In some embodiments, antibodies, antigen-binding fragments thereof, or compositions provided herein may be used alone or in combination with one or more additional therapeutic agents, e.g., other anti-cancer therapies. Thus, antibodies, antigen-binding fragments thereof and/or compositions described herein may be administered alone or in combination with one or more additional anti-cancer therapy. The latter can be administered before, after or simultaneously with the administration of the antibodies, antigen-binding fragments thereof and/or compositions described herein.

In some embodiments, the antibody or antigen-binding fragment of the disclosure and the one or more additional therapeutic agents are present in a combined composition. In some embodiments, the antibody or antigen-binding fragment or composition of the disclosure and the one or more additional therapeutic agents are administered separately. In some such embodiments, the antibody or antigen-binding fragment or composition of the disclosure may be used as an adjuvant therapy, e.g., to potentiate the one or more additional therapeutic agent. For example and without limitation, the antibody or antigen-binding fragment or composition of the disclosure may be used to potentiate ICI-blockade therapy, such as anti-PD1 or anti-PDL1 therapy, in a subject.

In some embodiments, a subject is in need of treatment by the methods provided herein and is selected for treatment based on this need. A subject in need of treatment is art-recognized, and includes subjects that have been identified as having a disease or condition (e.g., a tumor, an ICI-blockade resistant tumor), or having a symptom of such a disease or condition, or being at risk of such a disease or condition, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

As used herein, “treating” or “treatment” of a disease or condition refers, in some embodiments, to ameliorating at least one disease or condition (i.e., arresting or reducing the development of a disease or condition or at least one of the clinical symptoms thereof). In certain embodiments “treating” or “treatment” refers to ameliorating at least one physical parameter, such as e.g. tumor size, growth, or migration. In certain embodiments, “treating” or “treatment” refers to inhibiting or improving a disease or condition, either physically (e.g., stabilization of a discernible symptom), physiologically (e.g., stabilization of a physical parameter), or both. In certain embodiments, “treating” or “treatment” refers to delaying the onset (or recurrence) of a disease or condition. The term “treating” or “treatment” may refer to any indicia of success in the treatment or amelioration of a disease or condition, including any objective or subjective parameter such as abatement; remission; diminishing of symptoms or making the disease or condition more tolerable to the subject; improving a subject's physical or mental well-being, such as reducing pain experienced by the patient; and, in some situations additionally improving at least one parameter of a disease or condition.

As used herein, “preventing” or “prevention” is intended to refer at least to the reduction of the likelihood of, or the risk of, or susceptibility to acquiring a disease or disorder (i.e., causing at least one of the clinical symptoms of the disease not to develop in a patient that may be exposed to or predisposed to or at risk of the disease but does not yet experience or display symptoms of the disease). The term “prevention” or “preventing” is also used to describe the administration of a compound or composition described herein to a subject who is at risk of (or susceptible to) such a disease or condition. Subjects amenable to treatment for prevention of a disease or condition include individuals at risk of the disease or condition but not showing symptoms, as well as patients presently showing symptoms. In some embodiments, “prevention” or “preventing” is used to describe the administration of a compound or composition described herein to a subject who has been diagnosed with or treated for a disease or condition and is at risk of recurrence of the disease or condition.

In some embodiments, treatment or prevention are within the context of the present disclosure if there is a measurable difference between the performances of subjects treated using the methods provided herein as compared to members of a placebo group, historical control, or between subsequent tests given to the same subject.

The term “therapeutically effective amount” as used herein means that amount or dose of a compound or composition, upon single or multiple dose administration to a subject, which provides the desired effect (e.g., the desired biological or medicinal response, e.g., to ameliorate, lessen or prevent a disease, disorder or condition) in the subject being treated. In some embodiments, an effective amount is an amount or dose of an antibody or antigen-binding fragment or composition that prevents or treats a cancer in a subject, as described herein. The terms “effective amount” and “therapeutically effective amount” are used interchangeably herein.

It should be understood that the dosage or amount of a compound and/or composition used, alone or in combination with one or more active compounds to be administered, depends on the individual case and is, as is customary, to be adapted to the individual circumstances to achieve an optimum effect. Dosing and administration regimens are within the purview of the skilled artisan, and appropriate doses depend upon a number of factors within the knowledge of the ordinarily skilled physician, veterinarian, or researcher (e.g., see Wells et al. eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif (2000)). For example, dosing and administration regimens depend on the nature and the severity of the disorder to be treated, and also on the sex, age, weight and individual responsiveness of the human or animal to be treated, on the efficacy and duration of action of the compounds used, on whether the therapy is acute or chronic or prophylactic, and/or on whether other active compounds are administered in addition to the therapeutic molecule(s).

Thus the dose(s) of a compound or composition will vary depending upon a variety of factors including, but not limited to: the activity, biological and pharmacokinetic properties and/or side effects of the compound being used; the age, body weight, general health, gender, and diet of the subject; the time of administration, the route of administration, the rate of excretion, and any drug combination, if applicable; the effect which the practitioner desires the compound to have upon the subject; and the properties of the compound being administered (e.g. bioavailability, stability, potency, toxicity, etc). Such appropriate doses may be determined as known in the art. When one or more of the compounds of the disclosure is to be administered to humans, a physician may for example, prescribe a relatively low dose at first, subsequently increasing the dose until an appropriate response is obtained.

There are no particular limitations on the dose of each of the compounds for use in compositions provided herein. Exemplary doses include milligram or microgram amounts of the compound per kilogram of subject or sample weight (e.g., about 50 micrograms per kilogram to about 500 milligrams per kilogram, about 1 milligram per kilogram to about 100 milligrams per kilogram, about 1 milligram per kilogram to about 50 milligrams per kilogram, about 1 milligram per kilogram to about 10 milligrams per kilogram, or about 3 milligrams per kilogram to about 5 milligrams per kilogram). Additional exemplary doses include doses of about 5 to about 500 mg, about 25 to about 300 mg, about 25 to about 200 mg, about 50 to about 150 mg, or about 50, about 100, about 150 mg, about 200 mg or about 250 mg, and, for example, daily or twice daily, or lower or higher amounts.

In some embodiments, the dose range for adult humans is generally from 0.005 mg to 10 g/day orally. Tablets or other forms of presentation provided in discrete units may conveniently contain an amount of a compound (e.g., of Formula I, or in Table 1 or 2) which is effective at such dosage or as a multiple of the same, for instance, units containing mg to 500 mg, usually around 10 mg to 200 mg. A dosage unit (e.g., an oral dosage unit) can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g. 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, or 500 mg) of a compound described herein.

Administration of compounds and compositions provided herein can be carried out using known procedures, at dosages and for periods of time effective to achieve a desired purpose. Dosage regimens can be adjusted to provide the optimum therapeutic response. For example, several divided doses may be administered daily, or the dose may be proportionally reduced as indicated by the exigencies of the therapeutic situation. In some embodiments, a compound or composition is administered at an effective dosage sufficient to prevent or treat a cancer in a subject. Further, a compound or composition may be administered using any suitable route or means, such as without limitation via oral, parenteral, intravenous, intraperitoneal, intramuscular, sublingual, topical, or nasal administration, via inhalation, or via such other routes as are known in the art.

### Kits

Compound and compositions provided herein may be packaged as part of a kit, optionally including a container (e.g. packaging, a box, a vial, etc). The kit may be commercially used according to the methods described herein and may include instructions for use in such methods. Additional kit components may include acids, bases, buffering agents, inorganic salts, solvents, antioxidants, preservatives, or metal chelators. The additional kit components may be present as pure compositions, or as aqueous or organic solutions that incorporate one or more additional kit components. Any or all of the kit components optionally further comprise buffers.

### Cancer

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithlelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

In other embodiments, the cancer is glioblastoma, melanoma, Small-cell lung carcinoma (SCLC), neuroblastoma, sarcoma, glioma, or ovarian cancer.

In still other embodiments, the cancer is ovarian cancer, small cell lung cancer (SCLC), or melanoma.

### Cancer Therapies

In the methods of the present disclosure, therapeutic anti-GD2 antibodies and antigen-binding fragments thereof, and compositions thereof, can be used alone or can be used in combination with one or more additional anti-cancer therapy. Non-limiting examples of such additional anti-cancer therapies include chemotherapeutic agents, hormones, antiangiogens, radiolabelled compounds, surgery, cryotherapy, immunotherapy, cancer vaccines, immune cell engineering (e.g., CAR-T), and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, antibodies and antigen-binding fragments thereof of the present disclosure can be administered with a therapeutically effective dose of a chemotherapeutic agent. In other embodiments, antibodies and antigen-binding fragments thereof of the present disclosure are administered in conjunction with chemotherapy to enhance the activity and efficacy of the chemotherapeutic agent (e.g., use as an adjuvant therapy). The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, MR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments of methods of the disclosure, the anti-GD2 antibody or antigen-binding fragment thereof, or composition thereof, is administered in combination with one or more inhibitors of immune checkpoints (immune checkpoint inhibition therapy), such as PD1, PD-L1, and/or CD47 inhibitors.

Adoptive cell-based immunotherapies can be combined with the therapies of the present disclosure. Well-known adoptive cell-based immunotherapeutic modalities, include, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

The term “chimeric antigen receptor” or “CAR” refers to engineered T cell receptors (TCR) having a desired antigen specificity. T lymphocytes recognize specific antigens through interaction of the T cell receptor (TCR) with short peptides presented by major histocompatibility complex (MHC) class I or II molecules. For initial activation and clonal expansion, naive T cells are dependent on professional antigen-presenting cells (APCs) that provide additional co-stimulatory signals. TCR activation in the absence of co-stimulation can result in unresponsiveness and clonal anergy. To bypass immunization, different approaches for the derivation of cytotoxic effector cells with grafted recognition specificity have been developed. CARs have been constructed that consist of binding domains derived from natural ligands or antibodies specific for cell-surface components of the TCR-associated CD3 complex. Upon antigen binding, such chimeric antigen receptors link to endogenous signaling pathways in the effector cell and generate activating signals similar to those initiated by the TCR complex. Since the first reports on chimeric antigen receptors, this concept has steadily been refined and the molecular design of chimeric receptors has been optimized and routinely use any number of well-known binding domains, such as scFV and another protein binding fragments described herein.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NFkappaB signaling modulators, and immune checkpoint modulators, are used.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), 13C(indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any thereo, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 a antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well-known in the art (see, for example, U.S. Pat. Nos. 4,981,844 and 5,230,902 and PCT Publ. No. WO 2004/004483) can be used in the methods described herein.

Similarly, various agents or a combination thereof can be used to treat a cancer. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard V. J. et.al. Experimental Hematology, Volume 31, Number 6, June 2003, pp. 446-454(9); Herceg Z.; Wang Z.-Q. Mutation Research/Fundamental and Molecular Mechanisms of Mutagenesis, Volume 477, Number 1, 2 Jun. 2001, pp. 97-110(14)). Poly(ADP-ribose) polymerase 1 (PARP1) is a key molecule in the repair of DNA single-strand breaks (SSBs) (de Murcia J. et al. 1997. Proc Natl Acad Sci USA 94:7303-7307; Schreiber V, Dantzer F, Ame J C, de Murcia G (2006) Nat Rev Mol Cell Biol 7:517-528; Wang Z Q, et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant H E, et al. (2005) Nature 434:913-917; Farmer H, et al. (2005) Nature 434:917-921). The foregoing examples of chemotherapeutic agents are illustrative, and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

In some embodiments of methods of the disclosure, the additional or second anti-cancer therapy is a surgery, a chemotherapy, a cancer vaccine, a chimeric antigen receptor, a radiation therapy, an immunotherapy, a modulator of expression of immune checkpoint inhibitory proteins or ligands, or any combination thereof. In some embodiments, the immunotherapy is an immune checkpoint inhibition therapy. In some embodiments, the cancer therapy is avelumab, durvalumab, atezolizumab, BRAF/MEK inhibitor, a tyrosine kinase inhibitor, pembrolizumab, nivolumab, ipilimumab, or a combination thereof.

### Clincal Efficacy/Response to a Therapy

Clinical efficacy can be measured by any method known in the art. For example, the response to a therapy relates to any response of the cancer, e.g., a tumor, to the therapy, preferably to a change in tumor mass and/or volume after initiation of treatment. Tumor response may be assessed by comparing the size of a tumor after systemic intervention o the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of therapy, e.g., after a few hours, days, weeks or preferably after a few months. A typical endpoint for response assessment is upon termination of therapy or adjuvant therapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to a cancer therapy are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any cancer therapy. The outcome measurement may be pathologic response to therapy given in the adjuvant setting. Alternatively, outcome measures, such as overall survival and disease-free survival can be monitored over a period of time for subjects following the cancer therapy for whom biomarker measurement values are known. In certain embodiments, the same doses of anti-cancer agents are administered to each subject. In related embodiments, the doses administered are standard doses known in the art for anti-cancer agents. The period of time for which subjects are monitored can vary. For example, subjects may be monitored for at least 2, 4, 6, 8, 10, 12, 14, 16, 18, 20, 25, 30, 35, 40, 45, 55, or 60 months. Biomarker measurement threshold values that correlate to outcome of a cancer therapy can be determined using methods known in the art.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Example 1: Materials and Methods

Implantable Tumor Model: Mice Implanted with EL4 Tumor Cells

The animal protocol used was reviewed and approved by the Lady Davis Institute Animal Care Committee and animal experiments were performed according to the guidelines of the Canadian Council on Animal Care. Healthy wild-type female C57/B16 mice (10⁻¹² weeks of age, 19-20 g) were purchased from Harlan (Lachine, Quebec, Canada). A maximum of five mice per cage were kept in a 12-h dark-light cycle with food and water ad libitum. Mice were implanted subcutaneously with syngeneic EL4 tumor cells (75,000-150,000 cells depending on the experiment). Tumors were generally palpable after 3-4 days, and generally measurable quantitatively after 5-7 days. Therapies were applied at day 3 or 4 (when tumors were palpable) by intraperitoneal injection of the indicated treatment (anti-GD2 or anti-GD3 or anti-PD-1 mAbs or combinations thereof; versus vehicle or control irrelevant IgG, n=5 mice/group). Tumor growth were quantified and survival of the mice was monitored every 1-2 days starting at day 7.

Inducible Tumor Model: The BRAF^{V600E}/PTEN^{-/-} Genetic Model of Melanoma

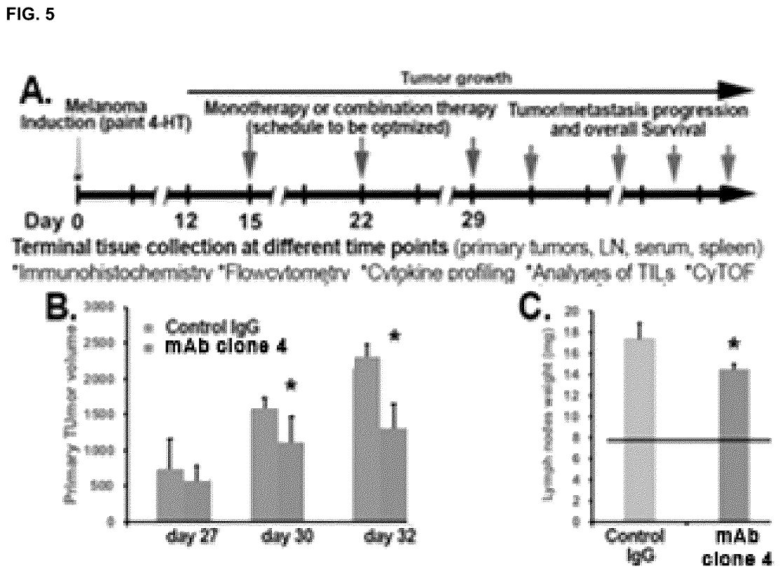

The BRAF^{V600E}/PTEN^{-/-} genetic model of melanoma is inducible by painting the skin of these engineered mice with a chemical called 4-HT. After the resulting induction of genetic injury, primary melanoma is first visible at day 12 (Dankort, D. et al., Nat Genet 41, 544-552, doi:10.1038/ng.356 (2009)). The BRAF^{V600E}/PTEN^{-/-} model is refractory to ICI-blockade therapy and therefore allows evaluation of adjuvanted therapies that may render a resistant tumor sensitive to standard-of-care therapies. or that may provide combinatorial (e.g., synergistic) effects therewith. The mAbs were tested in a therapeutic paradigm (n=5 mice/group). Treatment was 1× each at days 15, 22, and 29 (see FIG. 5A for a timeline of the experiment). The primary tumor was quantified over time and lymph node metastasis was quantified at day 37.

### ELISA

Gangliosides were isolated from equal volume of serum from each donor, using organic solvent extraction methods (see e.g., Example 10). Serum-equivalent volumes of analytes were immobilized in ELISA plates, and assayed with mAbs against GD2 or GD3. After incubation with secondary antibody, optical density (OD) was read at 450 nm. Negative values were set at optical density (OD)<0.15; low positive GD2 values at OD between 0.15-0.5 and high positive GD2 values at OD>0.5. Control purified native GD2 or GD3 (10 ng/well) were used as standard positive control. Wells with no primary were used as background. Each sample was assayed in triplicate, and each ELISA replicated at least 2-3 independent times.

### Flow Cytometry

2×10⁵ cells of EL4-GD2⁺, EL4-GD3⁺, and Jurkat cells were incubated for 20 min on ice with 2 mL mouse antisera (1:50 dilution) or with positive control anti-GD2 mAb (13 nM, 14G2a; Santa Cruz Biotechnology) or positive control anti-GD3 mAb (13 nM, R24; Abcam), followed by FITC-conjugated anti-mouse IgG secondary (1.8 nM, Sigma). Cells were studied immediately in a flow cytometer (Becton-Dickinson), and data analyzed using CellQuest software. Pre-bleed sera and normal mouse sera were used as negative controls. Jurkat cells (negative for GD2 and GD3) were used as negative control cells.

### Example 2: Binding Specificity of Monoclonal Antibodies

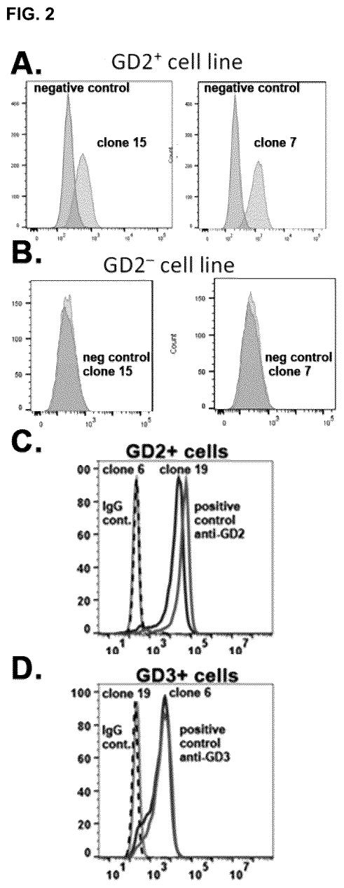

Binding specificity. Anti-human GD2 and GD3 antibodies were produced and characterized as described (see International PCT Application No. PCT/US2021/053503 (WO/2022/076364), which is incorporated herein by reference in its entirety). Binding assays were done using flow cytometry to characterize the binding specificities of the antibodies. Binding screens by flow cytometry showed binding to GD2 or to GD3 on the membrane of a panel of human and mouse cell lines expressing these targets, and no binding to control cells where the targets were absent. Examples of flow cytometry binding data are shown in FIG. 2(A)-(D).

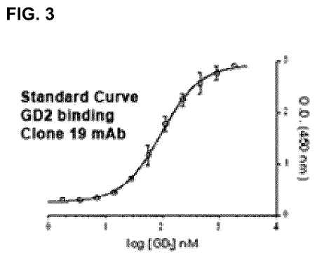

Binding tests were also done by ELISA, using pure GD2 or GD3 (or other gangliosides as controls) to demonstrate binding specificity for the mAbs. An example of an ELISA standard curve data is shown in FIG. 3.

The results of the binding assays are also summarized in Table 3, which shows flow cytometry and ELISA binding data for test mAbs, as indicated.

In sum, the results confirm that the 12 mAbs tested (see Tables 1 and 2 for their sequences) bind to the unique carbohydrate portion of GD2 or GD3 and are highly selective. In flow cytometry assays, each mAb bound only to the cell surface of cell lines expressing GD2 or expressing GD3 (mouse, rat or human) and there was no binding detected to cells known to be negative for GD2 or GD3.

TABLE 3
Flow cytrometry and ELISA binding data for test mAbs.

| | Flowcytometry binding | | ELISA binding | |
|---|---|---|---|---|
| | EL4 | EL4 | GD2 | GD3 |
| Clone # | GD2+ | GD3+ | target | target |
| 4 | 1162 | 2812 | + | + |
| 6 | 467 | 4863 | − | + |
| 7 | 2048 | 2930 | | |
| 8 | 6517 | 433 | | |
| 9 | 339 | 288 | + | + |
| 10 | 1147 | 751 | | |
| 13 | 670 | 968 | | |
| 14 | 300 | 321 | + | − |
| 15 | 4053 | 10754 | | |
| 17 | 2883 | 7096 | | |
| 18 | 396 | 370 | + | − |
| 19 | 10450 | 684 | + | − |
| Positive control | | | | |
| Anti-GD2 (Pharmingen) | 2128 | 282 | | |
| Anti-GD3 (Abcam) | 322 | 1247 | | |
| Negative control | | | | |
| Irrelevant Mouse Ig | 303 | 301 | − | − |

### Example 3: Functional Characterization of Monoclonal Antibodies

Cell cytotoxicity. In quantitative methods such as MTT assays, some mAbs can exhibit tumor cytotoxicity. Such in vitro assays test the killing of tumor cells expressing the antibody targets, by simple engagement of the mAb, in the absence of exogenous complement or any other immune effector.

Test mAbs were assayed for cytotoxicity on EL4 cells cultured in vitro. mAbs were evaluated in quantitative MTT assays. Results are shown in Table 4.

The results show that mAbs clone 4 and clone 17 selectively killed tumor cells in a dose-dependent manner, compared to IgG negative controls. In cellular controls none of these cytotoxic mAbs killed cells that do not express their targets. Two mAbs, clone 15 and clone 7, were not cytotoxic in vitro and did not kill cells. Therefore, the results show that, surprisingly, not all tested mAbs were cytotoxic in vitro. However, clone 15 and clone 7 may be able to kill tumors in vivo by inducing the action of effector mechanisms such as complement. Nevertheless the results indicate cytotoxicity of the majority of test antibodies suggesting they may be useful as anti-cancer therapeutic agents.

TABLE 4
MTT assay results for test mAbs at 50 nM.

EL4 cell survival assay (MTT)

| Clone # | OD reading | % survival |
|---|---|---|
| 4 | 0.21 ± 0.1 | 15.3 |
| 6 | 0.32 ± 0.2 | 23.4 |
| 7 | 1.41 ± 0.3 | 102.9 |
| 15 | 1.35 ± 0.2 | 98.5 |
| 17 | 0.34 ± 0.4 | 24.8 |
| 19 | 1.2 ± 0.4 | 87.6 |
| Negative control | | |
| Irrelevant Mouse Ig | 1.37 ± 0.3 | 100.0 |

Absence of pain-causing signatures, that lead to side effects. It has been reported that some anti-GD2 mAbs can cause serious side effects such as pain in patients, that cannot be blocked by morphine (Tong, W., et al., PLoS One 10, e0134255, doi:10.1371/journal.pone.0134255 (2015)). These pain-causing mAbs engage GD2 or GD3 on a small subset of neurons and induce pain-signals (e.g., activation of phospho-Src and phospho-NMDA-Receptor). Pain and other side effects are major clinical limitations.

Since biochemical activation of pSrc and pNR2B in neurons are predictive of pain, we tested our mAbs to see if they activate these pain signatures. The results showed that MAbs clone 4 and clone 17 do not activate pSrc or pNR2B. Combined with our in vivo finding that the mAbs clone 4 and clone 17 did not cause pain in mice when injected for therapeutic purposes, as discussed further hereinbelow (discussed further below), these results indicate strong therapeutic potential for the antibodies. We also tested the anti-GD2 mAb 3F8, which has been reported to cause pain in mice and humans (Tong, W., et al., PLoS One 10, e0134255, doi:10.1371/journal.pone.0134255 (2015)). In contrast to MAbs clone 4 and clone 17 (which did not activate the signatures of phospho-Src and phospho-NMDA-R), the anti-GD2 mAb 3F8 activated both pain signatures. Results are shown in Table 5. The observed absence of pain-signature side effects is a major advantage for clinical use, as it may resolve a safety limitation to the therapeutic index.

TABLE 5
mAbs do not activate pain signatures or cause pain in vivo.

| | | | Pain in vivo |
|---|---|---|---|
| | p-SRC pain | p-NR2B pain | (observed or |
| mAb tested | signature | signature | reported) |
| Negative control | | | |
| Irrelevant mouse IgG | − | − | − |
| Clone 4 | − | − | no |
| Clone 17 | − | − | no |
| BD Pharmingen 14G2a | ++ | ++ | Not known |
| mAb 3F8 | ++ | ++ | yes |

### Example 4: In Vivo Therapeutic Efficacy of mAbs, and Synergy with PD1-Targeted Therapies, in EL4 Tumor Model

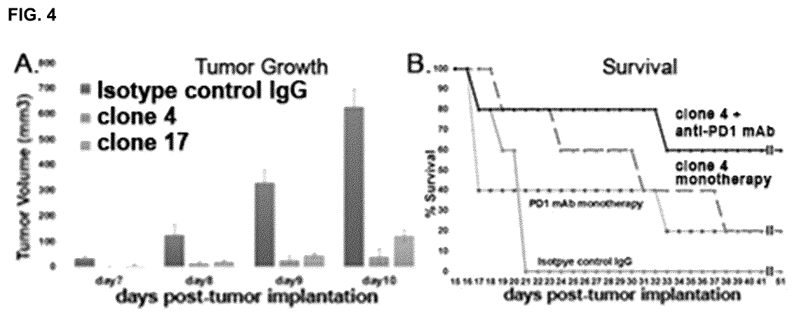

In vivo therapeutic efficacy of monotherapy with mAbs. We tested whether the mAbs could inhibit primary tumor growth in a mouse model. The mAbs clone 4 and clone 17 afforded a significant therapeutic benefit in vivo against implanted subcutaneous EL4 tumors, which are GD2⁺. A single therapeutic intervention (1×100 μg dose, IP, injected at day 3 after tumor implantation) resulted in a significant delay to primary tumor growth (FIG. 4A) and extension of life span (FIG. 4B).

The EL4 model (syngeneic to C57bl/6) is very aggressive and all control mice have to be euthanized by day 17-20. In contrast, a large percentage of anti-GD2 mAb-treated mice survived past day 30 (FIG. 4A). The extended life-span of the mice outlasted the estimated half-life of 2 weeks for the mAbs (FIG. 4B).

Synergy with PD1-targeted therapies. Further, the mAbs significantly potentiated PD1-targeted therapy. EL4 cells express high cell surface levels of the ligand PDL-1, which binds to PD1 checkpoint receptors in T cells to suppress immune activation. We examined the potential synergy between anti-PD1 and mAb therapies, using a therapeutic paradigm (single therapeutic intervention, at day 3 after tumor implantation). Mice received control isotype IgG, anti-PD1 mAb alone, mAb clone 4 alone, or a combination of anti-PD1 and mAb clone 4; each at 80 μg dose delivered i.p. (FIG. 4B). All control isotype IgG injected mice died by day 21, whereas the therapies afforded a significant survival. The monotherapy anti-PD1 mAb and the monotherapy mAb clone 4 were roughly comparable at extending life-span. Remarkably, the combination of mAb clone 4 and PD1 combination resulted in 60% long-term survivors past day 51.

In addition, the test mAbs clone 4 and clone 17 reduced lymph node metastasis of EL4 tumors in vivo. Lymph nodes were collected and weighed; increased lymph node weight reflects the presence of EL4 tumor cells. Clone 4 and clone 17 both reduced lymph node weight, indicating reduction of lymph node metastasis (n=4). Results are shown in Table 6.

TABLE 6
mAbs reduce lymph node metastasis of EL4 tumors in vivo.

| Treatment of | |
|---|---|
| mice in vivo | lymph node weight (mg) |
| Control IgG | 22.5 ± 4.0 |
| Clone 4 mAb | 15.3 ± 1.0 |
| Clone 17 mAb | 17.8 ± 1.1 |

In sum, compared to control, test mAbs afforded a significant delay to primary tumor growth, reduced metastasis, and extended life span. Moreover, they demonstrated a synergistic effect when combined with immune checkpoint inhibitor (ICI)-blockade therapies such as anti-PD1 antibody. Importantly, in all experiments the test mAbs did not cause any signs of pain in mice. There were no signs of pain or distress throughout the experiments.

We also evaluated the potential for optic nerve damage and retinopathy using objective and quantitative outcomes (optical tomography of the retina, performed as described)(Barcelona, P. F. et al., J Neurosci 36, 8826-8841, doi:10.1523/JNEUROSCI.4278-15.2016 (2016); Bai, Y. et al., J Biol Chem 285, 39392-39400 (2010); Bai, Y. et al. J Biol Chem 285, 39392-39400 (2010)). Test mAbs used at therapeutically effective doses caused no retinal damage, as measured by structural analyses using Optical Coherence Tomography. These in vivo safety data are consistent with these mAbs not inducing molecular signatures of neuronal activation and pain.

The results suggest that the side effects induced by other anti-GD2 mAbs are not inherent to the GD2 target, but rather they are a limitation inherent to the therapeutic agent, which binds GD2 as a ligand and activates neuronal signals leading to pain and excitotoxicity (Tong, W., et al., PLoS One 10, e0134255, doi:10.1371/journal.pone.0134255 (2015)).

### Example 5: In Vivo Therapeutic Efficacy of mAbs in the BRAF^{V600E}/PTEN^{-/-} Melanoma Model

The BRAF^{V600E}/PTEN^{-/-} genetic model of melanoma is inducible by painting the skin with a chemical called 4-HT. After the resulting induction of genetic injury, primary melanoma is first visible at day 12 (Dankort, D. et al., Nat Genet 41, 544-552, doi:10.1038/ng.356 (2009)). The BRAF^{V600E}/PTEN^{-/-} model is refractory to ICI-blockade therapy and therefore allows evaluation of adjuvanted therapies that may render a resistant tumor sensitive to standard-of-care therapies. or that may provide combinatorial (e.g., synergistic) effects therewith.

We tested the mAbs in a therapeutic paradigm using the BRAF^{V600E}/PTEN^{-/-} model (n=5 mice/group). Treatment was 1× each at days 15, 22, and 29 (see FIG. 5A for a timeline of the experiment). The primary tumor was quantified over time (FIG. 5B) and lymph node metastasis was quantified at day 37 (FIG. 5C). The results show that mAb clone 4 reduced the primary tumor volume (* p≤0.05) and lymph node metastasis (* p≤0.05) compared to control IgG.

Together, the above data indicate that the GD2-targeted therapies were effective in both the EL4 implantable model and BRAFV600E/PTEN−/− inducible model, arresting primary tumor growth and metastasis. Moreover, the GD2-targeted therapies potentiated the benefit of ICI-blockade therapy. The results provided hereinabove demonstrate surprisingly that the test mAbs are tumor-therapeutic but do not activate neuronal signals and do not cause side effects in vivo in mice.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and amino acid sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### Equivalents

Those skilled in the art will recognize, or be able to ascertain using no more than routine experimentation, many equivalents to the specific embodiments of the present invention described herein. Such equivalents are intended to be encompassed by the following claims.

What is claimed is:

1. A method for prevention or treatment of a cancer in a subject in need thereof, comprising administration of a therapeutically effective amount of an antibody specific for GD2 and/or GD3 or an antigen-binding fragment thereof to the subject, such that the cancer is prevented or treated.

2.-4. (canceled)

5. A The method of claim 1, further comprising administration of a second anti-cancer therapy to the subject, and wherein sensitivity of an ICI-resistant tumor to ICI-blockade therapy is increased in the subject.

6. The method of claim 1, wherein the antibody or the antigen-binding fragment thereof is administered in the form of a pharmaceutical composition comprising a pharmaceutically acceptable carrier.

7. The method of claim 5, wherein the second anti-cancer therapy is
an immune checkpoint inhibitor (ICI)-blockade therapy, such as an anti-PD1 or anti-PDL1 therapy, such as an antibody specific for PD1 or PDL1; or,
wherein the second anti-cancer therapy is chemotherapy, radiation therapy, hormone therapy, immunotherapy, targeted therapy, drug therapy, surgery or resection, or administration of another anti-cancer agent.

8.-10. (canceled)

11. A method of selecting and treating a subject suffering from an immune checkpoint inhibitor (ICI)-blockade therapy-resistant tumor or cancer, the method comprising the steps of:
(a) selecting the subject as having an ICI-blockade therapy-resistant tumor or cancer based on the presence of GD2 and/or GD3 on the tumor or cancer cells, or in a liquid biopsy from the subject; and
(b) administering to the selected subject a therapeutically effective amount of an antibody specific for GD2 and/or GD3 or an antigen-binding fragment thereof.

12. The method of claim 11, further comprising administering to the selected subject an immune checkpoint inhibitor (ICI)-blockade therapy, such as an anti-PD1 or anti-PDL1 therapy, such as an antibody specific for PD1 or PDL1.

13. A method of selecting a subject suffering from an immune checkpoint inhibitor (ICI)-blockade therapy-resistant tumor or cancer, the method comprising the steps of:
(a) obtaining a sample from the subject;
(b) assaying the sample for binding to an antibody specific for GD2 and/or GD3, wherein the sample that binds the antibody is respectively GD2+ and/or GD3+; and
(c) selecting the subject as suffering from an ICI-blockade therapy-resistant tumor or cancer if the sample is GD2+ and/or GD3+,
optionally wherein the sample comprises tumor or cancer cells or a biopsy, such as a liquid biopsy, such as blood or serum.

14. The method of claim 13, further comprising administering to the selected subject a therapeutically effective amount of an antibody specific for GD2 and/or GD3 or an antigen-binding fragment thereof.

15. The method of claim 1, wherein the antibody or the antigen-binding fragment thereof specifically binds to the carbohydrate portion of a ganglioside, wherein the ganglioside is (a) GD2, (b) GD3, or (c) GD2 and GD3.

16. (canceled)

17. The method of claim 1, wherein the antibody is a monoclonal antibody specific for GD2, GD3, or GD2 and GD3,
wherein the antibody or antigen-binding fragment thereof, comprises:
a) a combination of a heavy chain CDR1, CDR2, and CDR3 as set forth in Table 1, or a variant sequence thereof which differs by only one or two amino acids, or which has at least or about 85% sequence identity thereto; and/or,
b) a combination of a light chain CDR1, CDR2, and CDR3 as set forth in Table 1, or a variant sequence thereof which differs by only one or two amino acids, or which has at least or about 85% sequence identity thereto; or,
wherein the antibody or antigen-binding fragment thereof, comprises:
c) a VH sequence as set forth in Table 2, or a variant sequence thereof which differs by only one or two amino acids, or which has at least or about 85% sequence identity thereto; and/or
d) a VL sequence as set forth in Table 2, or a variant sequence thereof which differs by only one or two amino acids, or which has at least or about 85% sequence identity thereto.

18.-20. (canceled)

21. The method of claim 1, wherein the antibody or antigen-binding fragment thereof is administered in the form of an antibody drug conjugate (ADC) wherein the antibody or the antigen-binding fragment thereof is conjugated to an anti-cancer drug such as a chemotherapeutic agent.

22.-25. (canceled)

26. The method of claim 17, wherein the antibody or antigen-binding fragment thereof, comprises six CDR amino acid sequences selected from:
a) SEQ ID NOs: 2, 4, 6, 8, 10, and 12 (clone 4), or a variant sequence thereof which differs by only one or two amino acids, or which has at least or about 85% sequence identity thereto;
b) SEQ ID NOs: 14, 16, 18, 20, 22, and 24 (clone 6), or a variant sequence thereof which differs by only one or two amino acids, or which has at least or about 85% sequence identity thereto;
c) SEQ ID NOs: 26, 28, 30, 32, 34, and 36 (clone 7), or a variant sequence thereof which differs by only one or two amino acids, or which has at least or about 85% sequence identity thereto;
d) SEQ ID NOs: 38, 40, 42, 44, 46, and 48 (clone 8), or a variant sequence thereof which differs by only one or two amino acids, or which has at least or about 85% sequence identity thereto;
e) SEQ ID NOs: 50, 52, 54, 56, 58, and 60 (clone 9), or a variant sequence thereof which differs by only one or two amino acids, or which has at least or about 85% sequence identity thereto;
f) SEQ ID NOs: 62, 64, 66, 68, 70, and 72 (clone 10), or a variant sequence thereof which differs by only one or two amino acids, or which has at least or about 85% sequence identity thereto;
g) SEQ ID NOs: 74, 76, 78, 80, 82, and 84 (clone 13), or a variant sequence thereof which differs by only one or two amino acids, or which has at least or about 85% sequence identity thereto;
h) SEQ ID NOs: 86, 88, 90, 92, 94, and 96 (clone 14), or a variant sequence thereof which differs by only one or two amino acids, or which has at least or about 85% sequence identity thereto;
i) SEQ ID NOs: 98, 100, 102, 104, 106, and 108 (clone 15), or a variant sequence thereof which differs by only one or two amino acids, or which has at least or about 85% sequence identity thereto;
j) SEQ ID NOs: 110, 112, 114, 116, 118, and 120 (clone 17), or a variant sequence thereof which differs by only one or two amino acids, or which has at least or about 85% sequence identity thereto;
k) SEQ ID NOs: 122, 124, 126, 128, 130, and 132 (clone 18), or a variant sequence thereof which differs by only one or two amino acids, or which has at least or about 85% sequence identity thereto; and
l) SEQ ID NOs: 134, 136, 138, 140, 142, and 144 (clone 19), or a variant sequence thereof which differs by only one or two amino acids, or which has at least or about 85% sequence identity thereto.

27. The method of claim 17, wherein the anti-GD2 monoclonal antibody or antigen-binding fragment thereof, comprises six CDR amino acid sequences selected from:
i) SEQ ID NOs: 2, 4, 6, 8, 10, and 12 (clone 4), or a variant sequence thereof which differs by only one or two amino acids, or which has at least or about 85% sequence identity thereto;
ii) SEQ ID NOs: 14, 16, 18, 20, 22, and 24 (clone 6), or a variant sequence thereof which differs by only one or two amino acids, or which has at least or about 85% sequence identity thereto;
iii) SEQ ID NOs: 110, 112, 114, 116, 118, and 120 (clone 17), or a variant sequence thereof which differs by only one or two amino acids, or which has at least or about 85% sequence identity thereto; and
iv) SEQ ID NOs: 134, 136, 138, 140, 142, and 144 (clone 19), or a variant sequence thereof which differs by only one or two amino acids, or which has at least or about 85% sequence identity thereto; or,
wherein the antibody or antigen-binding fragment thereof, comprises the VH and VL amino acid sequences selected from:
a) SEQ ID NOs: 146 and 148, or a variant sequence thereof which differs by only one or two amino acids, or which has at least or about 85% sequence identity thereto;
b) SEQ ID NOs: 150 and 152, or a variant sequence thereof which differs by only one or two amino acids, or which has at least or about 85% sequence identity thereto;
c) SEQ ID NOs: 154 and 156, or a variant sequence thereof which differs by only one or two amino acids, or which has at least or about 85% sequence identity thereto;
d) SEQ ID NOs: 158 and 160, or a variant sequence thereof which differs by only one or two amino acids, or which has at least or about 85% sequence identity thereto;
e) SEQ ID NOs: 162 and 164, or a variant sequence thereof which differs by only one or two amino acids, or which has at least or about 85% sequence identity thereto;
f) SEQ ID NOs: 166 and 168, or a variant sequence thereof which differs by only one or two amino acids, or which has at least or about 85% sequence identity thereto;
g) SEQ ID NOs: 170 and 172, or a variant sequence thereof which differs by only one or two amino acids, or which has at least or about 85% sequence identity thereto;
h) SEQ ID NOs: 174 and 176, or a variant sequence thereof which differs by only one or two amino acids, or which has at least or about 85% sequence identity thereto;
i) SEQ ID NOs: 178 and 180, or a variant sequence thereof which differs by only one or two amino acids, or which has at least or about 85% sequence identity thereto;
j) SEQ ID NOs: 182 and 184, or a variant sequence thereof which differs by only one or two amino acids, or which has at least or about 85% sequence identity thereto;
k) SEQ ID NOs: 186 and 188, or a variant sequence thereof which differs by only one or two amino acids, or which has at least or about 85% sequence identity thereto; and
l) SEQ ID NOs: 190 and 192, or a variant sequence thereof which differs by only one or two amino acids, or which has at least or about 85% sequence identity thereto; or,
wherein the antibody or antigen-binding fragment thereof, comprises the VH and VL amino acid sequences selected from:
v) SEQ ID NOs: 146 and 148, or a variant sequence thereof which differs by only one or two amino acids, or which has at least or about 85% sequence identity thereto;
vi) SEQ ID NOs: 150 and 152, or a variant sequence thereof which differs by only one or two amino acids, or which has at least or about 85% sequence identity thereto;
vii) SEQ ID NOs: 182 and 184, or a variant sequence thereof which differs by only one or two amino acids, or which has at least or about 85% sequence identity thereto; and
viii) SEQ ID NOs: 190 and 192, or a variant sequence thereof which differs by only one or two amino acids, or which has at least or about 85% sequence identity thereto.

28.-29. (canceled)

30. The method of claim 1, wherein:
a) the antibody or antigen-binding fragment thereof is chimeric, humanized, composite, murine, camelid, llama, or human; and/or
b) the antibody or antigen-binding fragment thereof comprises an immunoglobulin heavy chain constant domain selected from the group consisting of IgG, IgG1, IgG2, IgG2A, IgG2B, IgG3, IgG4, IgA, IgM, IgD, and IgE constant domains.

31. The method of claim 1, wherein the antibody or antigen-binding fragment thereof is detectably labeled or conjugated, comprises an effector domain, comprises an Fc domain, and/or is selected from the group consisting of Fv, F(ab′)2, Fab′, dsFv, scFv, sc(Fv)2, and diabodies fragments.

32. The method of claim 1, wherein the antibody or antigen-binding fragment thereof comprises an immunoglobulin heavy and/or light chain selected from the immunoglobulin heavy and light chain sequences listed in Table 2.

33. The method of claim 1, wherein an isolated nucleic acid molecule that encodes the antibody or antigen-binding fragment thereof is administered to the subjects, or,
wherein a vector comprising an isolated nucleic acid molecule that encodes the antibody or antigen-binding fragment thereof is administered to the subject.

34. (canceled)

35. The method of claim 1, wherein the cancer is selected from the group consisting of neuroblastoma, lymphoma, leukemia, melanoma, glioma, small cell lung cancer, breast carcinoma, ovarian cancer, soft tissue sarcomas, osteosarcoma, Ewing's sarcoma, desmoplastic round cell tumor, rhabdomyosarcoma, retinoblastoma, non-small cell lung cancer, renal cell cancer, Wilms tumor, prostate cancer, gastric cancer, endometrial cancer, pancreatic cancer, and colon cancer.

36.-41. (canceled)

42. The method of claim 1, wherein the subject has cancer, has a predisposition for cancer, or is at risk of having cancer or a cancer recurrence; optionally wherein the subject is a human; optionally wherein the subject has a tumor which is GD2+ and/or GD3+; or, wherein the tumor or cancer in the subject is resistant to immune checkpoint inhibitor (ICI)-blockade therapy.

43.-44. (canceled)

45. The method of claim 1, wherein said admistration is via a parenteral, oral, transdermal or topical, transmucosal, or rectal route of administration, where said parenteral route of administration is intravenous, intramuscular, subcutaneous, or intraperitoneal administration.

46. (canceled)


SEQUENCE LISTING (192 sequences)

SEQ ID NO: 1 - DNA, 24 bp, synthetic construct
  source 1..24: mol_type = other DNA
ggctacacatttaccaggtactgg

SEQ ID NO: 2 - protein, 8 aa, synthetic construct
GYTFTRYW

SEQ ID NO: 3 - DNA, 24 bp, synthetic construct
  source 1..24: mol_type = other DNA
atttatcctggaaatagtgatact

SEQ ID NO: 4 - protein, 8 aa, synthetic construct
IYPGNSDT

SEQ ID NO: 5 - DNA, 27 bp, synthetic construct
  source 1..27: mol_type = other DNA
gcaagatccgatggtcctatggactac

SEQ ID NO: 6 - protein, 9 aa, synthetic construct
ARSDGPMDY

SEQ ID NO: 7 - DNA, 30 bp, synthetic construct
  source 1..30: mol_type = other DNA
gaaagtgttgataattatggcatcagtttt

SEQ ID NO: 8 - protein, 10 aa, synthetic construct
ESVDNYGISF

SEQ ID NO: 9-10 - skipped (2 entries)

SEQ ID NO: 11 - DNA, 27 bp, synthetic construct
  source 1..27: mol_type = other DNA
cagcaaagtaaggaggttccgttcacg

SEQ ID NO: 12 - protein, 9 aa, synthetic construct
QQSKEVPFT

SEQ ID NO: 13 - DNA, 15 bp, synthetic construct
  source 1..15: mol_type = other DNA
acctatggaatgagc

SEQ ID NO: 14 - protein, 5 aa, synthetic construct
TYGMS

SEQ ID NO: 15 - DNA, 51 bp, synthetic construct
  source 1..51: mol_type = other DNA
tggataaacacatatactggagtgccaacatatggtgatgacttcaaggga

SEQ ID NO: 16 - protein, 17 aa, synthetic construct
WINTYTGVPTYGDDFKG

SEQ ID NO: 17 - DNA, 27 bp, synthetic construct
  source 1..27: mol_type = other DNA
tggttacgccaccatgctatggactac

SEQ ID NO: 18 - protein, 9 aa, synthetic construct
WLRHHAMDY

SEQ ID NO: 19 - DNA, 33 bp, synthetic construct
  source 1..33: mol_type = other DNA
aaggccagtgagaatgtggttacttatgtttcc

SEQ ID NO: 20 - protein, 11 aa, synthetic construct
KASENVVTYVS

SEQ ID NO: 21 - DNA, 21 bp, synthetic construct
  source 1..21: mol_type = other DNA
ggggcatccaaccggtacact

SEQ ID NO: 22 - protein, 7 aa, synthetic construct
GASNRYT

SEQ ID NO: 23 - DNA, 27 bp, synthetic construct
  source 1..27: mol_type = other DNA
ggacagggttacagctatccgtacacg

SEQ ID NO: 24 - protein, 9 aa, synthetic construct
GQGYSYPYT

SEQ ID NO: 25 - DNA, 24 bp, synthetic construct
  source 1..24: mol_type = other DNA
ggattcacttttagtgacgcctgg

SEQ ID NO: 26 - protein, 8 aa, synthetic construct
GFTFSDAW

SEQ ID NO: 27 - DNA, 30 bp, synthetic construct
  source 1..30: mol_type = other DNA
attagaaacaaagctaataatcatgcgaca

SEQ ID NO: 28 - protein, 10 aa, synthetic construct
IRNKANNHAT

SEQ ID NO: 29 - DNA, 30 bp, synthetic construct
  source 1..30: mol_type = other DNA
accaggcgacatgattcctactttgactac

SEQ ID NO: 30 - protein, 10 aa, synthetic construct
TRRHDSYFDY

SEQ ID NO: 31 - DNA, 18 bp, synthetic construct
  source 1..18: mol_type = other DNA
caggatgtggatactgct

SEQ ID NO: 32 - protein, 6 aa, synthetic construct
QDVDTA

SEQ ID NO: 33-34 - skipped (2 entries)

SEQ ID NO: 35 - DNA, 27 bp, synthetic construct
  source 1..27: mol_type = other DNA
cagcaatatcgcagctatcctctcacg

SEQ ID NO: 36 - protein, 9 aa, synthetic construct
QQYRSYPLT

SEQ ID NO: 37 - DNA, 24 bp, synthetic construct
  source 1..24: mol_type = other DNA
ggctacacatttaccagttactgg

SEQ ID NO: 38 - protein, 8 aa, synthetic construct
GYTFTSYW

SEQ ID NO: 39 - DNA, 24 bp, synthetic construct
  source 1..24: mol_type = other DNA
atttatcctggaaaaagtggtact

SEQ ID NO: 40 - protein, 8 aa, synthetic construct
IYPGKSGT

SEQ ID NO: 41 - DNA, 27 bp, synthetic construct
  source 1..27: mol_type = other DNA
acaagatccgatggtcctatggactac

SEQ ID NO: 42 - protein, 9 aa, synthetic construct
TRSDGPMDY

SEQ ID NO: 43 - DNA, 30 bp, synthetic construct
  source 1..30: mol_type = other DNA
gagagtgttgataattatgacattagtttt

SEQ ID NO: 44 - protein, 10 aa, synthetic construct
ESVDNYDISF

SEQ ID NO: 45-46 - skipped (2 entries)

SEQ ID NO: 47 - DNA, 27 bp, synthetic construct
  source 1..27: mol_type = other DNA
cagcaaagtaaggaggttccgtacacg

SEQ ID NO: 48 - protein, 9 aa, synthetic construct
QQSKEVPYT

SEQ ID NO: 49 - DNA, 15 bp, synthetic construct
  source 1..15: mol_type = other DNA
gactacaacatggac

SEQ ID NO: 50 - protein, 5 aa, synthetic construct
DYNMD

SEQ ID NO: 51 - DNA, 51 bp, synthetic construct
  source 1..51: mol_type = other DNA
gatattaatcctaacaatggtggtactatctacaaccagaagttcaagggc

SEQ ID NO: 52 - protein, 17 aa, synthetic construct
DINPNNGGTIYNQKFKG

SEQ ID NO: 53 - DNA, 36 bp, synthetic construct
  source 1..36: mol_type = other DNA
tcggggatctactatgattacgcctggtttccttac

SEQ ID NO: 54 - protein, 12 aa, synthetic construct
SGIYYDYAWFPY

SEQ ID NO: 55 - DNA, 33 bp, synthetic construct
  source 1..33: mol_type = other DNA
agtgcaagtcagggcattagcaattatttaaac

SEQ ID NO: 56 - protein, 11 aa, synthetic construct
SASQGISNYLN

SEQ ID NO: 57 - DNA, 21 bp, synthetic construct
  source 1..21: mol_type = other DNA
tacacatcaagtttacactca

SEQ ID NO: 58 - protein, 7 aa, synthetic construct
YTSSLHS

SEQ ID NO: 59 - DNA, 27 bp, synthetic construct
  source 1..27: mol_type = other DNA
cagcagtatagtaagcttcctcctacg

SEQ ID NO: 60 - protein, 9 aa, synthetic construct
QQYSKLPPT

SEQ ID NO: 61 - DNA, 15 bp, synthetic construct
  source 1..15: mol_type = other DNA
gactacaacatggac

SEQ ID NO: 62 - protein, 5 aa, synthetic construct
DYNMD

SEQ ID NO: 63 - DNA, 51 bp, synthetic construct; identical to SEQ ID NO: 51
  source 1..51: mol_type = other DNA

SEQ ID NO: 64 - protein, 17 aa, synthetic construct
DINPNNGGTIYNQKFKG

SEQ ID NO: 65 - DNA, 36 bp, synthetic construct; identical to SEQ ID NO: 53
  source 1..36: mol_type = other DNA

SEQ ID NO: 66 - protein, 12 aa, synthetic construct
SGIYYDYAWFPY

SEQ ID NO: 67 - DNA, 33 bp, synthetic construct; identical to SEQ ID NO: 55
  source 1..33: mol_type = other DNA

SEQ ID NO: 68 - protein, 11 aa, synthetic construct
SASQGISNYLN

SEQ ID NO: 69 - DNA, 21 bp, synthetic construct
  source 1..21: mol_type = other DNA
tacacatcaagtttacactca

SEQ ID NO: 70 - protein, 7 aa, synthetic construct
YTSSLHS

SEQ ID NO: 71 - DNA, 27 bp, synthetic construct
  source 1..27: mol_type = other DNA
cagcagtatagtaagcttcctcctacg

SEQ ID NO: 72 - protein, 9 aa, synthetic construct
QQYSKLPPT

SEQ ID NO: 73 - DNA, 15 bp, synthetic construct
  source 1..15: mol_type = other DNA
gactatgaaatgcac

SEQ ID NO: 74 - protein, 5 aa, synthetic construct
DYEMH

SEQ ID NO: 75 - DNA, 51 bp, synthetic construct
  source 1..51: mol_type = other DNA
gctattgatcctgaaactggtggtactgcctacaatcagaagttcaagggc

SEQ ID NO: 76 - protein, 17 aa, synthetic construct
AIDPETGGTAYNQKFKG

SEQ ID NO: 77 - DNA, 18 bp, synthetic construct
  source 1..18: mol_type = other DNA
agctgggacggagactac

SEQ ID NO: 78 - protein, 6 aa, synthetic construct
SWDGDY

SEQ ID NO: 79 - DNA, 33 bp, synthetic construct
  source 1..33: mol_type = other DNA
aaggccagtcagaatgtgggtactaatgtagcc

SEQ ID NO: 80 - protein, 11 aa, synthetic construct
KASQNVGTNVA

SEQ ID NO: 81 - DNA, 21 bp, synthetic construct
  source 1..21: mol_type = other DNA
tcggcatcctaccggtacagt

SEQ ID NO: 82 - protein, 7 aa, synthetic construct
SASYRYS

SEQ ID NO: 83 - DNA, 27 bp, synthetic construct
  source 1..27: mol_type = other DNA
cagcaatataacagctatccattcacg

SEQ ID NO: 84 - protein, 9 aa, synthetic construct
QQYNSYPFT

SEQ ID NO: 85 - DNA, 15 bp, synthetic construct
  source 1..15: mol_type = other DNA
gactacaacatggac

SEQ ID NO: 86 - protein, 5 aa, synthetic construct
DYNMD

SEQ ID NO: 87 - DNA, 51 bp, synthetic construct; identical to SEQ ID NO: 51
  source 1..51: mol_type = other DNA

SEQ ID NO: 88 - protein, 17 aa, synthetic construct
DINPNNGGTIYNQKFKG

SEQ ID NO: 89 - DNA, 36 bp, synthetic construct; identical to SEQ ID NO: 53
  source 1..36: mol_type = other DNA

SEQ ID NO: 90 - protein, 12 aa, synthetic construct
SGIYYDYAWFPY

SEQ ID NO: 91 - DNA, 33 bp, synthetic construct; identical to SEQ ID NO: 55
  source 1..33: mol_type = other DNA

SEQ ID NO: 92 - protein, 11 aa, synthetic construct
SASQGISNYLN

SEQ ID NO: 93 - DNA, 21 bp, synthetic construct
  source 1..21: mol_type = other DNA
tacacatcaagtttacactca

SEQ ID NO: 94 - protein, 7 aa, synthetic construct
YTSSLHS

SEQ ID NO: 95 - DNA, 27 bp, synthetic construct
  source 1..27: mol_type = other DNA
cagcagtatagtaagcttcctcctacg

SEQ ID NO: 96 - protein, 9 aa, synthetic construct
QQYSKLPPT

SEQ ID NO: 97 - DNA, 24 bp, synthetic construct
  source 1..24: mol_type = other DNA
ggattcacttttagtgacgcctgg

SEQ ID NO: 98 - protein, 8 aa, synthetic construct
GFTFSDAW

SEQ ID NO: 99 - DNA, 30 bp, synthetic construct; identical to SEQ ID NO: 27
  source 1..30: mol_type = other DNA

SEQ ID NO: 100 - protein, 10 aa, synthetic construct
IRNKANNHAT

SEQ ID NO: 101 - DNA, 30 bp, synthetic construct
  source 1..30: mol_type = other DNA
accgggcgacatgattcctactttgactac

SEQ ID NO: 102 - protein, 10 aa, synthetic construct
TGRHDSYFDY

SEQ ID NO: 103 - DNA, 18 bp, synthetic construct
  source 1..18: mol_type = other DNA
cagggcattagcaattat

SEQ ID NO: 104 - protein, 6 aa, synthetic construct
QGISNY

SEQ ID NO: 105-106 - skipped (2 entries)

SEQ ID NO: 107 - DNA, 27 bp, synthetic construct
  source 1..27: mol_type = other DNA
cagcagtatagtaagcttcctcctacg

SEQ ID NO: 108 - protein, 9 aa, synthetic construct
QQYSKLPPT

SEQ ID NO: 109 - DNA, 24 bp, synthetic construct
  source 1..24: mol_type = other DNA
ggctacaccttcaccagctactgg

SEQ ID NO: 110 - protein, 8 aa, synthetic construct
GYTFTSYW

SEQ ID NO: 111 - DNA, 24 bp, synthetic construct
  source 1..24: mol_type = other DNA
atttatcctggtagtggtagtact

SEQ ID NO: 112 - protein, 8 aa, synthetic construct
IYPGSGST

SEQ ID NO: 113 - DNA, 51 bp, synthetic construct
  source 1..51: mol_type = other DNA
gcaagccaccgatttgattactacggtagtagctactatgctatggactac

SEQ ID NO: 114 - protein, 17 aa, synthetic construct
ASHRFDYYGSSYYAMDY

SEQ ID NO: 115 - DNA, 18 bp, synthetic construct
  source 1..18: mol_type = other DNA
caggacatttgcaattat

SEQ ID NO: 116 - protein, 6 aa, synthetic construct
QDICNY

SEQ ID NO: 117-118 - skipped (2 entries)

SEQ ID NO: 119 - DNA, 27 bp, synthetic construct
  source 1..27: mol_type = other DNA
caacagggtaatacgcttccgctcacg

SEQ ID NO: 120 - protein, 9 aa, synthetic construct
QQGNTLPLT

SEQ ID NO: 121 - DNA, 24 bp, synthetic construct
  source 1..24: mol_type = other DNA
ggatacacgttcactgacttccac

SEQ ID NO: 122 - protein, 8 aa, synthetic construct
GYTFTDFH

SEQ ID NO: 123 - DNA, 24 bp, synthetic construct
  source 1..24: mol_type = other DNA
attaatcctaacaatggtggtact

SEQ ID NO: 124 - protein, 8 aa, synthetic construct
INPNNGGT

SEQ ID NO: 125 - DNA, 30 bp, synthetic construct
  source 1..30: mol_type = other DNA
gtaagagaaatctactttggctttgactac

SEQ ID NO: 126 - protein, 10 aa, synthetic construct
VREIYFGFDY

SEQ ID NO: 127 - DNA, 18 bp, synthetic construct
  source 1..18: mol_type = other DNA
caggacatttgcaattat

SEQ ID NO: 128 - protein, 6 aa, synthetic construct
QDICNY

SEQ ID NO: 129-130 - skipped (2 entries)

SEQ ID NO: 131 - DNA, 27 bp, synthetic construct
  source 1..27: mol_type = other DNA
caacagggtaatacgcttccgctcacg

SEQ ID NO: 132 - protein, 9 aa, synthetic construct
QQGNTLPLT

SEQ ID NO: 133 - DNA, 24 bp, synthetic construct
  source 1..24: mol_type = other DNA
ggatacacattcactaaatacacc

SEQ ID NO: 134 - protein, 8 aa, synthetic construct
GYTFTKYT

SEQ ID NO: 135 - DNA, 24 bp, synthetic construct
  source 1..24: mol_type = other DNA
attaatcctaacaatggtggtact

SEQ ID NO: 136 - protein, 8 aa, synthetic construct
INPNNGGT

SEQ ID NO: 137 - DNA, 21 bp, synthetic construct
  source 1..21: mol_type = other DNA
acaagcaagtcctttgactac

SEQ ID NO: 138 - protein, 7 aa, synthetic construct
TSKSFDY

SEQ ID NO: 139 - DNA, 15 bp, synthetic construct
  source 1..15: mol_type = other DNA
tcaagtgtaagtaac

SEQ ID NO: 140 - protein, 5 aa, synthetic construct
SSVSN

SEQ ID NO: 141-142 - skipped (2 entries)

SEQ ID NO: 143 - DNA, 27 bp, synthetic construct
  source 1..27: mol_type = other DNA
caacaaaggagtggttacccattcacg

SEQ ID NO: 144 - protein, 9 aa, synthetic construct
QQRSGYPFT

SEQ ID NO: 145 - DNA, 348 bp, synthetic construct
  source 1..348: mol_type = other DNA
caagtacagctggaggagtctgggactgtgctggcaaggcctggggcttcagtgaagatgtcctgcaagacttctggctacacatttaccaggtactggatgcactgggtaaaacagaggcctggacagggtctggaatggataggggctatttatcctggaaatagtgatactacctacaaccagaagttcaagggcaaggccaaactgactgcagtcacatccgccaccaatgcctacatggaagtaagcagcctgacaaatgaggactctgcggtctattactgtgcaagatccgatggtcctatggactactggggtcaaggaacctcagtcaccgtctcctca

SEQ ID NO: 146 - protein, 116 aa, synthetic construct
QVQLEESGTVLARPGASVKMSCKTSGYTFTRYWMHWVKQRPGQGLEWIGAIYPGNSDTTYNQKFKGKAKLTAVTSATNAYMEVSSLTNEDSAVYYCARSDGPMDYWGQGTSVTVSS

SEQ ID NO: 147 - DNA, 326 bp, synthetic construct
  source 1..326: mol_type = other DNA
gacattgtactgacccaatctccagcttctttggctgtgtctctagggcagagggccaccatttcctgcagagccagcgaaagtgttgataattatggcatcagttttatgaactggttccaacagaaaccaggacagccacccaaactcctcatctatgctgcatccaatcaaggatccggggtccctgccaggtttagtggcagtgggtctgggacagacttcagtctcaacatccatcctatggaggaggatgatactgcaatgtatttctgtcagcaaagtaaggaggttccgttcacgttcggaggggggaccaagctgga

SEQ ID NO: 148 - protein, 108 aa, synthetic construct
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPFTFGGGTKL

SEQ ID NO: 149 - DNA, 354 bp, synthetic construct
  source 1..354: mol_type = other DNA
cagatccagttggtacaatctggacctgagctgaagaagcctggagagacagtcaagatctcctgcaaggcttctggatattccttcacaacctatggaatgagctgggtgaaacaggctccaggaaagggtttaaagtggatgggctggataaacacatatactggagtgccaacatatggtgatgacttcaagggacggtttgccttctctttggaaacctctaccagcactgcctatttgcagatcaacaacctcaaaaatgacgacacggcttcatatttctgtgcaagatggttacgccaccatgctatggactactggggtcaaggaacctcagtcaccgtctcctca

SEQ ID NO: 150 - protein, 118 aa, synthetic construct
QIQLVQSGPELKKPGETVKISCKASGYSFTTYGMSWVKQAPGKGLKWMGWINTYTGVPTYGDDFKGRFAFSLETSTSTAYLQINNLKNDDTASYFCARWLRHHAMDYWGQGTSVTVSS

SEQ ID NO: 151 - DNA, 321 bp, synthetic construct
  source 1..321: mol_type = other DNA
aacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaa

SEQ ID NO: 152 - protein, 107 aa, synthetic construct
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 153 - DNA, 357 bp, synthetic construct
  source 1..357: mol_type = other DNA
caggttcagctggagcagtctggaggagccttggtgcaacctggaggatccatgaaactctcttgtgctgcctctggattcacttttagtgacgcctggatggactgggtccgccagtctccagaaaaggggcttgagtgggttgctgaaattagaaacaaagctaataatcatgcgacatactatgctgagtctgtgaaagggaggttcaccatctcaagagatgattccaaaaatagtgtctacctgcaaatgaacaacttaagagctgaagacactggcatttattattgtaccaggcgacatgattcctactttgactactggggccaaggcaccactctcacagtctcctca

SEQ ID NO: 154 - protein, 119 aa, synthetic construct
QVQLEQSGGALVQPGGSMKLSCAASGFTFSDAWMDWVRQSPEKGLEWVAEIRNKANNHATYYAESVKGRFTISRDDSKNSVYLQMNNLRAEDTGIYYCTRRHDSYFDYWGQGTTLTVSS

SEQ ID NO: 155 - DNA, 324 bp, synthetic construct
  source 1..324: mol_type = other DNA
gacattgtgatgacccagtctcacaaattcatgtccacatcagtaggagacagggtcagcatcacctacaaggccagtcaggatgtggatactgctgtagcctggtatcaacagaaaccagggcaatctcctaaactactgatttactgggcatccacccggcacactggagtccctgatcgcttcacaggcagtggatctgggacagatttcactctcaccattaacaatgtgcagtctgaagacttggcagattatttctgtcagcaatatcgcagctatcctctcacgttcggtgctgggaccaagctggaactgaaacgg

SEQ ID NO: 156 - protein, 108 aa, synthetic construct
DIVMTQSHKFMSTSVGDRVSITYKASQDVDTAVAWYQQKPGQSPKLLIYWASTRHTGVPDRFTGSGSGTDFTLTINNVQSEDLADYFCQQYRSYPLTFGAGTKLELKR

SEQ ID NO: 157 - DNA, 348 bp, synthetic construct
  source 1..348: mol_type = other DNA
gaagtaaagctgcaggagtctgggactgagctggcaaggcctggggcttcagtgaagatgtcctgcaagacttctggctacacatttaccagttactggatgcactgggtaaaacagaggcctggacagggtctggaatggataggggctatttatcctggaaaaagtggtactacctacaaccagaagttcaagggcaaggccaaactgactgcagtcacatccgccagcactgcctacatggaactcagcagcctgacaaatgaggactctgcggtctattactgtacaagatccgatggtcctatggactactggggtcaaggaacctcagtcaccgtctcctca

SEQ ID NO: 158 - protein, 116 aa, synthetic construct
EVKLQESGTELARPGASVKMSCKTSGYTFTSYWMHWVKQRPGQGLEWIGAIYPGKSGTTYNQKFKGKAKLTAVTSASTAYMELSSLTNEDSAVYYCTRSDGPMDYWGQGTSVTVSS

SEQ ID NO: 159 - DNA, 336 bp, synthetic construct
  source 1..336: mol_type = other DNA
gacattgtgctgacccaatctccagcttctttggctgtgtctctagggcagagggccaccatctcctgcagagccagcgagagtgttgataattatgacattagttttatgaactggttccaacagaaaccaggacagccacccaaactcctcatctatgctgcatccaaccaaggatccggggtccctgccaggttcagtggcagtgggtctgggacagacttcagtctcaacatccatcctatggaggaggatgatactgcaatgtatttctgtcagcaaagtaaggaggttccgtacacgttcggaggggggaccaagctggaaataaaacgg

SEQ ID NO: 160 - protein, 112 aa, synthetic construct
DIVLTQSPASLAVSLGQRATISCRASESVDNYDISFMNWFQQKPGQPPKLLIYAASNQGSGVPARFSGSGSGTDFSLNIHPMEEDDTAMYFCQQSKEVPYTFGGGTKLEIKR

SEQ ID NO: 161 - DNA, 363 bp, synthetic construct
  source 1..363: mol_type = other DNA
gaggtccagctgcaacagtctggacctgagctggtgaagcctggggcttcagtgaagataccctgcaaggcttctggatacacattcactgactacaacatggactgggtgaaacagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagatcggggatctactatgattacgcctggtttccttactggggccaagggactctggtcactgtctctgca

SEQ ID NO: 162 - protein, 121 aa, synthetic construct
EVQLQQSGPELVKPGASVKIPCKASGYTFTDYNMDWVKQSHGKSLEWIGDINPNNGGTIYNQKFKGKATLTVDKSSSTAYMELRSLTSEDTAVYYCARSGIYYDYAWFPYWGQGTLVTVSA

SEQ ID NO: 163 - DNA, 321 bp, synthetic construct
  source 1..321: mol_type = other DNA
gatatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttcctcctacgttcggtgctgggaccaagctggagctgaaa

SEQ ID NO: 164 - protein, 107 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPPTFGAGTKLELK

SEQ ID NO: 165 - DNA, 364 bp, synthetic construct
  source 1..364: mol_type = other DNA
gaggtccagctgcaacagtctggacctgagctggtgaagcctggggcttcagtgaagataccctgcaaggcttctggatacacattcactgactacaacatggactgggtgaaacagagccatggaaagagccttgagtggattggagatattaatcctaacaatggtggtactatctacaaccagaagttcaagggcaaggccacattgactgtagacaagtcctccagcacagcctacatggagctccgcagcctgacatctgaggacactgcagtctattactgtgcaagatcggggatctactatgattacgcctggtttccttactggggccaagggactctggtcactgtctctgcag

SEQ ID NO: 166 - protein, 121 aa, synthetic construct; identical to SEQ ID NO: 162

SEQ ID NO: 167 - DNA, 322 bp, synthetic construct
  source 1..322: mol_type = other DNA
gatatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttcctcctacgttcggtgctgggaccaagctggagctgaaac

SEQ ID NO: 168 - protein, 107 aa, synthetic construct; identical to SEQ ID NO: 164

SEQ ID NO: 169 - DNA, 345 bp, synthetic construct
  source 1..345: mol_type = other DNA
caggttcaactgcagcagtctggggctgagctggtgaggcctggggcttcagtgacgctgtcctgcaaggcttcgggctacacatttactgactatgaaatgcactgggtgaagcagacacctgtgcatggcctggaatggattggagctattgatcctgaaactggtggtactgcctacaatcagaagttcaagggcaaggccatactgactgcagacaaatcctccagcacagcctacatggagctccgcagcctgacatctgaggactctgccgtctattactgtacaagaagctgggacggagactactggggccaaggcaccactctcacagtctcctca

SEQ ID NO: 170 - protein, 115 aa, synthetic construct
QVQLQQSGAELVRPGASVTLSCKASGYTFTDYEMHWVKQTPVHGLEWIGAIDPETGGTAYNQKFKGKAILTADKSSSTAYMELRSLTSEDSAVYYCTRSWDGDYWGQGTTLTVSS

SEQ ID NO: 171 - DNA, 321 bp, synthetic construct
  source 1..321: mol_type = other DNA
gacattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccattcacgttcggctcggggacaaagttggaaataaaa

SEQ ID NO: 172 - protein, 107 aa, synthetic construct
DIVMTQSQKFMSTSVGDRVSVTCKASQNVGTNVAWYQQKPGQSPKALIYSASYRYSGVPDRFTGSGSGTDFTLTISNVQSEDLAEYFCQQYNSYPFTFGSGTKLEIK

SEQ ID NO: 173 - DNA, 364 bp, synthetic construct; identical to SEQ ID NO: 165
  source 1..364: mol_type = other DNA

SEQ ID NO: 174 - protein, 121 aa, synthetic construct; identical to SEQ ID NO: 162

SEQ ID NO: 175 - DNA, 322 bp, synthetic construct; identical to SEQ ID NO: 167
  source 1..322: mol_type = other DNA

SEQ ID NO: 176 - protein, 107 aa, synthetic construct; identical to SEQ ID NO: 164

SEQ ID NO: 177 - DNA, 357 bp, synthetic construct
  source 1..357: mol_type = other DNA
caagttcagctgcaggagtctggaggagccttggtgcaacctggaggatccatgaaactctcttgtgctgcctctggattcacttttagtgacgcctggatggactgggtccgccagtctccagaaaaggggcttgagtgggttgctgaaattagaaacaaagctaataatcatgcgacatactatgctgagtctgtgaaagggaggttcaccatctcaagagatgattccaaaaatagtgtctacctgcaaatgaacaacttaagagctgaggacactggcatttattactgtaccgggcgacatgattcctactttgactactggggccaaggcaccactctcacagtctcctca

SEQ ID NO: 178 - protein, 119 aa, synthetic construct
QVQLQESGGALVQPGGSMKLSCAASGFTFSDAWMDWVRQSPEKGLEWVAEIRNKANNHATYYAESVKGRFTISRDDSKNSVYLQMNNLRAEDTGIYYCTGRHDSYFDYWGQGTTLTVSS

SEQ ID NO: 179 - DNA, 324 bp, synthetic construct
  source 1..324: mol_type = other DNA
gatatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttcctcctacgttcggtgctgggaccaagctggagctgaaacgg

SEQ ID NO: 180 - protein, 108 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPPTFGAGTKLELKR

SEQ ID NO: 181 - DNA, 372 bp, synthetic construct
  source 1..372: mol_type = other DNA
gaggtccagctggaggagtctggggctgagcttgtgaagcctggggcttcggtgaagatgtcctgtaaggcttctggctacaccttcaccagctactggataacctgggtgaagcagaggcctggacaaggccttgagtggattggagatatttatcctggtagtggtagtactaactacaatgagaagttcaagagcaaggccacactgactgtagacacatcctccagcacaacctacatgcagctcagcagcctgacatctgaggactctgcggtctattactgtgcaagccaccgatttgattactacggtagtagctactatgctatggactactggggtcaaggaacctcagtcaccgtctcctca

SEQ ID NO: 182 - protein, 124 aa, synthetic construct
EVQLEESGAELVKPGASVKMSCKASGYTFTSYWITWVKQRPGQGLEWIGDIYPGSGSTNYNEKFKSKATLTVDTSSSTTYMQLSSLTSEDSAVYYCASHRFDYYGSSYYAMDYWGQGTSVTVSS

SEQ ID NO: 183 - DNA, 324 bp, synthetic construct
  source 1..324: mol_type = other DNA
gatatccagatgacacagactacatcctccctgtctgcctctctgggagacagagtcaccatcagttgcagggcaagtcaggacatttgcaattatttaaactggtatcagcagaaaccagatggaccttttaaactcctgatcttctacacatcaagattacactcaggagtcccatcaaggttcagtggcagtgggtctggaacagattattctctcaccattagcaacctggagcaagaagatattgccacttacttttgccaacagggtaatacgcttccgctcacgttcggtgctgggaccaagctggagctgaaacgg

SEQ ID NO: 184 - protein, 108 aa, synthetic construct
DIQMTQTTSSLSASLGDRVTISCRASQDICNYLNWYQQKPDGPFKLLIFYTSRLHSGVPSRFSGSGSGTDYSLTISNLEQEDIATYFCQQGNTLPLTFGAGTKLELKR

SEQ ID NO: 185 - DNA, 351 bp, synthetic construct
  source 1..351: mol_type = other DNA
gaagtacagctggaggagtctggacctgagctggtgaagcctgggacttcagtgaagatatcctgtaaggcttctggatacacgttcactgacttccacattaactgggtgaaacagagccatggaaagaaccttgagtggattggagatattaatcctaacaatggtggtactaactacaaccagaaattcaagggcaaggccacattgattgttgacaagtcttccagcgcagcctacatggagctccgcagcctgacatctgaggactctgcagtctattattgtgtaagagaaatctactttggctttgactactggggccaaggcaccactctcacagtctcctca

SEQ ID NO: 186 - protein, 117 aa, synthetic construct
EVQLEESGPELVKPGTSVKISCKASGYTFTDFHINWVKQSHGKNLEWIGDINPNNGGTNYNQKFKGKATLIVDKSSSAAYMELRSLTSEDSAVYYCVREIYFGFDYWGQGTTLTVSS

SEQ ID NO: 187 - DNA, 324 bp, synthetic construct; identical to SEQ ID NO: 183
  source 1..324: mol_type = other DNA

SEQ ID NO: 188 - protein, 108 aa, synthetic construct; identical to SEQ ID NO: 184

SEQ ID NO: 189 - DNA, 342 bp, synthetic construct
  source 1..342: mol_type = other DNA
gaagtgaagctggaggagtctggacctgagctggtgaagcctggggcttcagtgaagatatcctgcaagacttctggatacacattcactaaatacaccatgcactgggtgaagcagagccatggaaagagccttgagtggattggagatattaatcctaacaatggtggtactaactacaaccagaagttcaagggcacggccacattgactgtacacaagtcctccaccacagcctacatggagctccgcagcctgacatctgaggattctgcagtctattactgtacaagcaagtcctttgactactggggccaaggcaccactctcacagtctcctca

SEQ ID NO: 190 - protein, 114 aa, synthetic construct
EVKLEESGPELVKPGASVKISCKTSGYTFTKYTMHWVKQSHGKSLEWIGDINPNNGGTNYNQKFKGTATLTVHKSSTTAYMELRSLTSEDSAVYYCTSKSFDYWGQGTTLTVSS

SEQ ID NO: 191 - DNA, 321 bp, synthetic construct
  source 1..321: mol_type = other DNA
caaattgttctcacccagtctccagcaatcatgtctgcatctccaggggagaaggtcaccataacctgcagtgccagctcaagtgtaagtaacatacactggttccagcagaagccaggcacttttcccaaactctggatttatagcacatccaccctggcttctggagtccctggtcgcttcagtggcagtggatctgggacctcttactctctcacaatcagccgaatgggggctgaagatgctgccacttattactgccaacaaaggagtggttacccattcacgttcggctcggggacaaagttggaaataaaacgg

SEQ ID NO: 192 - protein, 107 aa, synthetic construct
QIVLTQSPAIMSASPGEKVTITCSASSSVSNIHWFQQKPGTFPKLWIYSTSTLASGVPGRFSGSGSGTSYSLTISRMGAEDAATYYCQQRSGYPFTFGSGTKLEIKR